(19) United States
(12) Patent Application Publication
(10) Pub. No.: US 2026/0070895 A1
(43) Pub. Date: Mar. 12, 2026

(54) CRYSTALLINE FORMS

(71) Applicant: QUIMATRYX, S.L., Guipúzcoa, San Sebastián (ES)

(72) Inventors: Yosu Ion VARA SALAZAR, Guipúzcoa, San Sebastián (ES); Eneko ALDABA AREVALO, Guipúzcoa, San Sebastián (ES); Tamara BELLO IGLESIAS, Guipúzcoa, San Sebastián (ES); Laureano SIMON BUELA, Guipúzcoa, San Sebastián (ES)

(21) Appl. No.: 18/858,116

(22) PCT Filed: May 4, 2022
(86) PCT No.: PCT/EP2022/062061

§ 371 Date: Oct. 18, 2024

(30) Foreign Application Priority Data
Apr. 19, 2022 (CN) 202210409814.5

(51) Int. Cl. C07D 405/04; C12N 9/80; C12N 11/02

(52) U.S. Cl. CPC C07D 405/04; C12N 9/80; C12N 11/02; C12Y 305/01098

(57) ABSTRACT

The present invention relates to novel crystalline forms of a compound of formula I, or an adduct thereof:

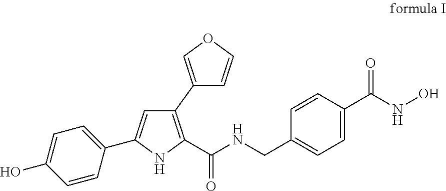 [SMILES: O=C(NO)c1ccc(CNC(=O)c2[nH]c(-c3ccc(O)cc3)cc2-c2ccoc2)cc1]

It further relates to methods of preparing such novel crystalline forms, to pharmaceutical compositions comprising them, methods of preparing the pharmaceutical compositions, and uses and medical treatments using the novel crystalline forms.

### CROSS-REFERENCE TO RELATED APPLICATIONS

This application is a 371 National Phase Entry of PCT/EP2022/062061, filed May 4, 2022, which claims priority to CN Application No. 202210409814.5, filed Apr. 19, 2022, the entire contents of which are incorporated by reference herein.

### FIELD OF THE INVENTION

This invention generally pertains to novel crystalline forms of a certain histone deacetylase inhibitor and adducts thereof. The present invention also pertains to the pharmaceutical compositions comprising the novel crystalline forms, methods for the preparation of the novel crystalline forms and pharmaceutical compositions, as well as their use in the treatment of diseases such as proliferative or autoimmune diseases.

### BACKGROUND

### Histone Deacetylase (HDAC)

Histone deacetylases (HDAC) constitute an interesting therapeutic target for the treatment of cancer (cf. P. A. Marks et al., Nature Rev. Cancer, 2001, 1, 194; J. E. Bolden et al., Nature Rev. Drug Discov., 2006, 5, 769; P. Gallinari et al., Cell Res., 2007, 17, 195; K. B. Glaser, Biochem. Pharmacol., 2007, 74, 659; L. Pan et al., Cell. Mol. Immunol., 2007, 4, 337; M. Haberland et al., Nature Rev. Genetics, 2009, 10, 32; Y. Zhang et al., Curr. Med. Chem., 2008, 15, 2840; S. Ropero and M. Esteller, Mol. Oncol., 2007, 1, 19) and other diseases such as those related to central nervous system, such as autoimmune diseases (cf. A. G. Kazantsev and L. M. Thompson, Nature Rev. Drug Discov., 2006, 7, 854).

Several families of HDAC inhibitors (HDACis) have been designed, whose general structures can be found in different reviews (cf. A. Villar-Garea and M. Esteller, Int. J. Cancer, 2004, 112, 171; T. A. Miller et al., J. Med. Chem., 2003, 46, 5097; T. Suzuki and N. Miyata, Curr. Med. Chem., 2005, 12, 2867; M. Paris et al., J. Med. Chem., 2008, 51, 1505). The general structure of these inhibitors consists of a cyclic structure, a spacer and a chelating group capable of binding to the Zn (II) cation of the active centre of the different HDAC isoforms that belong to the class I (HDAC1, HDAC2, HDAC3 and HDAC8), class II (HDAC4, HDAC5, HDAC6, HDAC7, HDAC9 and HDAC10) and class IV (HDAC11).

The mechanism of action of the HDAC inhibitors is explained by their antagonist properties against histone deacetylases involved in the regulation of processes related to apoptosis, cell growth, tumour progression, cancer metastasis, cell adhesion and others. These properties prevent the binding of HDACs to their natural ligands, which can be histones or cytoplasmic proteins such as tubulin, as well as their normal catalytic activation, namely the deacetylation of ε-N-acetyl lysine residues present in these proteins.

Despite having a similar inhibition mode, occasionally some selectivity in the inhibition of different HDAC isoforms has been observed (cf. J. C. Wong et al., J. Am. Chem. Soc., 2003, 125, 5586; G. Estiu et al., J. Med. Chem., 2008, 51, 2898). The mentioned selectivity is of therapeutic interest (cf. K. V. Butler and A. P. Kozikowski, Curr. Pharm. Design, 2008, 14, 505; T. C. Karagiannis and A. El-Osta, Leukemia, 2007, 21, 61).

### HDAC Inhibitors

One important class of HDAC inhibitors are trisubstituted pyrrolic derivatives connected with the chelating groups through aromatic and heteroaromatic groups, as described for example, in WO 2011/039353. These compounds have been shown to be effective in the treatment of cancer (cf. WO 2011/039353).

In addition, there compounds have been shown to be effective in the treatment of several autoimmune diseases. For example, these compounds have been shown to be effective in animal models of autoimmune hepatitis and autoimmune encephalomyelitis (cf. WO 2018/087082).

An especially promising compound is 3-(3-furyl)-N-{4-[(hydroxyamino)carbonyl]benzyl}-5-(4-hydroxyphenyl)-1H-pyrrole-2-carboxamide (referred to herein as QTX125).

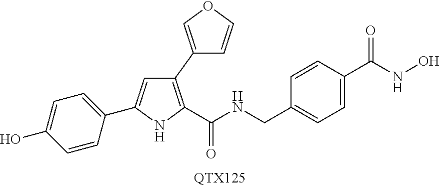

QTX125 is a highly selective and highly potent HDAC 6 inhibitor. It has shown high antitumoral efficacy in mantle cell lymphoma (cf. Perez-Salvia, M. et al., Haematologica, 2018; 103: e540), lung cancer and pancreatic cancer xenograft murine models. QTX125 has also shown high efficacy in two different multiple sclerosis mice models (cf. WO 2018/087082).

However, hydroxamic acids such as QTX125 are known to have very low solubility in water (cf. Patre, S. et al., International Conference on Environment and BioScience IPCBEE, 2011, vol. 21) and to dissolve QTX125 in aqueous solution it is normally necessary to employ high pH values. QTX125 also demonstrates physical and chemical instability in solution.

Consequently, there remains a need in the art to provide novel forms of QTX125 which may be particularly, but not exclusively, useful for pharmaceutical formulations. Especially, novel forms of QTX125 which contain high concentrations of QTX125 at physiological pH which are stable and have low toxicity are particularly desirable.

Several patents and publications are cited herein to describe and disclose the invention and the state of the art to which the invention pertains more fully. Full citations for these references are provided herein. Each of these references is incorporated herein by reference in its entirety into the present disclosure.

### SUMMARY OF THE INVENTION

The present inventors have developed crystalline forms of QTX125 and adducts thereof, which help to address the practical problems outlined above.

In an aspect, the present invention relates to a crystalline form of a compound of formula I or an adduct thereof:

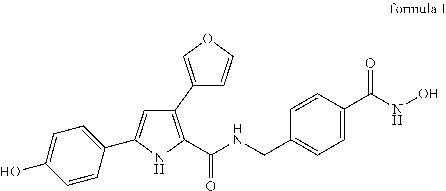 [SMILES: O=C(NO)c1ccc(CNC(=O)c2[nH]c(-c3ccc(O)cc3)cc2-c2ccoc2)cc1]

which is characterized by a powder x-ray diffraction (PXRD) pattern having peaks at 2θ=20.4°, 21.8°, 22.0°, 22.7° and 23.9° (±0.3° 2θ).

The compound of formula I is also referred to herein as QTX125. The compound of formula I (QTX125) is 3-(3-Furyl)-N-{4-[(hydroxyamino)carbonyl]benzyl}-5-(4-hydroxy phenyl)-1H-pyrrole-2-carboxamide.

Another aspect of the present invention relates to a pharmaceutical composition comprising a crystalline form of a compound of formula I or an adduct thereof of the present invention.

Another aspect of the present invention relates to an in vitro complex, comprising a crystalline form of a compound of formula I or an adduct thereof of the present invention.

Further aspects of the present invention relate to processes for the preparation of a crystalline form of a compound of formula I or an adduct thereof of the present invention, and crystalline forms of a compound of formula I or an adduct thereof of the present invention obtainable by those processes.

Further aspects of the present invention relate to methods of preparing a pharmaceutical composition comprising a crystalline form of a compound of formula I or an adduct thereof of the present invention, and pharmaceutical compositions obtainable by such methods.

Another aspect of the present invention relates to a crystalline form of a compound of formula I or and adduct thereof of the present invention, for use as a medicament.

Another aspect of the present invention relates to the use of a crystalline form of a compound of formula I or and adduct thereof of the present invention, in the preparation of a medicament.

According to another aspect, the present invention relates to a method of treating a mammal which comprises administering to a patient needing such treatment, a therapeutically effective amount of at least one crystalline form of a compound of formula (I) or an adduct thereof of the present invention.

According to a particular embodiment, a crystalline form of a compound of formula (I) or an adduct thereof of the present invention is useful for the treatment of various types of cancer by restricting tumor growth or other processes that stop the development of primary or metastatic tumors, through the inhibition of certain histone deacetylases.

According to a particular embodiment, the adduct of a compound of formula I of the present invention is an adduct with lysine, particularly a 1:2 adduct with L-lysine.

### BRIEF DESCRIPTION OF THE FIGURES

Embodiments and experiments illustrating the principles of the invention will now be discussed with reference to the accompanying figures:

FIG. 1A shows a thermogravimetric analysis (TGA) graph for crude QTX125. The delta Y indicated is 8.252%. FIG. 1B shows an overlay of graphs produced from differential scanning calorimetry (DSC)—in dark grey—and TGA—in pale grey—of crude QTX125. The delta Y indicated is 8.252%. FIG. 1C shows a powder x-ray diffraction (PXRD) pattern of crude QTX125.

FIG. 2A shows a PXRD pattern of QTX125 Form 2 isolated via extractive purification and water slurry. FIG. 2B shows a graph produced from DSC of Form 2 alone. The indicated onset is 213.97° C., the indicated peak is at 221.72° C. and has a peak height of −7.1369 mW. The area is −356.666 mJ and delta H is −178.3332 J/g. FIG. 2C shows a graph produced from DSC of Form 2 (pale grey, lower) overlaid with that of crude QTX125 (dark grey, upper). The indicated onset, peak, peak height, area and delta H values are as shown in FIG. 2B. For the crude QTX125, the corresponding peak and peak height values indicated are 210.55° C. and 13.8539 mW. Also indicated are 181.08° C. and 22.6070 mW which are values corresponding to a minor endothermic peak of the crude QTX125. FIG. 2D shows overlaid graphs produced from TGA (dark grey) and DSC (pale grey) of Form 2. The indicated delta Y is 12.255%.

FIG. 3A shows a PXRD pattern of Form 2 isolated via crystallisation (uppermost) overlaid with that of Form 2 isolated via extractive purification and water slurry (lowermost). FIG. 3B shows a graph produced from DSC of Form 2 isolated by crystallisation. The indicated onset is 234.28° C., the peak is 237.25° C., peak height is −30.1217 mW, area is −545.835 mJ and delta H is −227.4312 J/g. Also indicated are 233.20° C. and −11.8407 mW which correspond to a small endothermic event.

FIG. 4A shows the PXRD pattern of Form 2 isolated (i) via first scale-up crystallisation (uppermost); (ii) via trial crystallization (middle); and of crude QTX125 (lowermost). FIG. 4B shows graphs produced from DSC of Form 2 isolated (i) via a first scale-up reaction (palest grey, lowermost); and (ii) via trial crystallisation (mid-grey, middle); and of crude QTX125 (dark grey, uppermost). The indicated peaks are, respectively, at (i) 235.88° C. (peak height is −4.8278 mW); (ii) 236.84° C. (peak height is 0.5430 mW); and (iii) 210.31° C. (peak height is 13.8495 mW). Also indicated are 180.53° C. with 22.66056 mW—corresponding to a minor endothermic peak of the crude QTX125—as well as 229.49° C. with 19.3510 mW and 229.89° C. (peak height is 18.1257 mW)—corresponding to minor endothermic events of the Form 2. FIG. 4C shows an overlay of graphs produced from DSC (pale grey) and TGA (dark grey, upper) of Form 2 isolated via a first scale-up reaction; and a TGA graph of Form 2 QTX125 isolated via trial crystallization (pale grey, lower). For Form 2 first scale-up the indicated delta Y is 8.500% and for Form 2 trial crystallization it is 9.229%. The indicated peak is at 235.88° C. (peak height −4.8292 mW). Also noted are 229.89° C. and 18.1258 mW, corresponding to a minor endothermic event of the Form 2.

FIG. 5A shows PXRD patterns of Form 2 isolated via a first scale-up (lowermost) and of Form 2 isolated via a second scale-up (uppermost). FIG. 5B shows an overlay of graphs produced from DSC (pale grey) and TGA (dark grey) of Form 2, isolated via a second scale-up reaction. The indicated delta Y is 8.384%, the peak indicated is at 238.66° C. (peak height is −1.1993 mW). Also noted are 228.78° C. and 19.2562 mW, corresponding to a minor endothermic event of the Form 2.

FIG. 6A shows solubility profiles of QTX125 Form 2 in six aqueous solutions as discussed in Example 5 (briefly: phosphate buffer at pH 3.5; phosphate buffer at pH 6.5; acetate buffer at pH 4.5; citro-phosphate buffer at pH 4.5; NaCl at 0.9% w/v, and water). FIG. 6B shows a profile expansion of the solubility profiles of QTX125 Form 2 in five of those solutions (per FIG. 6A except for the water).

FIG. 7A shows solubility profiles of QTX125 1:2 L-Lysine adduct in six aqueous buffers (the same as indicated for FIG. 6A). FIG. 7B shows a profile expansion of the solubility profiles of the adduct in four of those buffers (per FIG. 7A except for NaCl at 0.9% w/v, and water).

FIG. 8 Solubility profiles of two QTX125 (Form 2, black, and 1:2 L-Lysine adduct, grey) entities in deionised water.

FIG. 9 PXRD pattern for crystalline 1:2 L-Lysine adduct of QTX125.

FIG. 10 Overlaid graphs produced from TGA and DSC of crystalline 1:2 L-lysine adduct of QTX125. (a) denotes a TGA delta Y=0.850%; (b) denotes a TGA delta Y=0.593%; (c) denotes a DSC peak at 95.38° C. and 21.2157 mW; (d) denotes a TGA delta Y=4.685%; (e) denotes a DSC peak at 155.85° C. and 22.7664 mW; (f) denotes a DSC peak at 167.51° C. and 22.3195 mW; and (g) denotes a DSC peak at 184.40° C. and 21.7741 mW.

### DETAILED DESCRIPTION

### Definitions

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The term “Cₓ-C_{y} alkyl” refers to a linear or branched hydrocarbon chain consisting of carbon and hydrogen atoms, containing no unsaturation, having from x to y carbon atoms. For example, the term “C₁-C₄ alkyl” refers to a linear or branched hydrocarbon chain consisting of carbon and hydrogen atoms, containing no unsaturation, having from 1 to 4 carbon atoms, preferably between 1 and 3 (“C₁-C₃ alkyl”), and which is attached to the rest of the molecule through a single bond, including for example and in a non-limiting sense, methyl, ethyl, n-propyl, i-propyl, n-butyl, t-butyl etc.

The term “about” preceding a stated value indicates that the value may have an uncertainty of ±20%, preferably ±10%, ±5%, ±2%, ±1% of the stated value.

The term “room temperature” refers to the ambient temperature of a typical laboratory, which is typically between 20° C. and 30° C., preferably around 25° C., at atmospheric pressure.

The term “dry” refers to a component e.g. crystalline form or composition which has been subjected to drying. Optionally, this may refer to a solid material with a residual water content of less than 10%, preferably less than 8%, preferably less than 5%, preferably from about 0.1% to about 5%. The residual water content may be determined using a Karl Fischer Titration.

The term “injection” refers to any form of injection known to a skilled person in the art such as subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal. Injection may refer to an infusion process (e.g. sustained administration) as well as bolus (discreate) administration.

The term “pharmaceutically acceptable salts” refers to salts which, when administered to the recipient, can provide (directly or indirectly) a compound as described in the present document. “Pharmaceutically acceptable” preferably refers to compositions and molecular entities that are physiologically tolerable and do not usually produce an allergic reaction or a similar unfavourable reaction such as gastric disorders, dizziness, and suchlike, when administered to a human or animal. Preferably, the term “pharmaceutically acceptable” means it is approved by a regulatory agency of a state or federal government or is included in the U.S. Pharmacopoeia or other generally recognized pharmacopoeia for use in animals, and more particularly in humans.

The term “adduct” is a product of a direct addition of two or more distinct molecules. The result is a single reaction product containing all atoms of all components. For example, the adduct of QTX125 and L-lysine as discussed further herein is believed to be produced by an interaction between the L-lysine and QTX125.

Adducts can be prepared by methods known in the art. Note that the non-pharmaceutically acceptable adducts also fall within the scope of the invention because they can be useful in preparing pharmaceutically acceptable adducts.

The compounds of the invention also seek to include compounds that differ only in the presence of one or more isotopically enriched atoms. For example, compounds having the present structures except for the replacement of a hydrogen by a deuterium or tritium, or the replacement of a carbon by a carbon enriched in ¹¹C, ¹³C or ¹⁴C or a ¹⁵N enriched nitrogen are within the scope of this invention.

The term “treatment” or “treating” refers to administration of a compound or a pharmaceutical composition of the invention to improve or eliminate the disease or one or more symptoms associated with the disease. The term “prevention” or “prevent” includes reducing the risk of the disease appearing or developing.

If not indicated otherwise “%” refers to weight-%.

By “±0.3° 2θ” we mean that the peaks describing the PXRD pattern may differ by up to 0.3° 2θ from the values mentioned. In some embodiments, the peaks may differ by up to 0.2° 2θ, such as by 0.1° 2θ or by 0.0° 2θ from the mentioned values.

The present invention has several advantageous features, including those listed below.

The crystalline forms of QTX125 and adducts thereof as described herein have various unexpected properties.

Firstly, they have improved photostability compared to other forms, including an amorphous form.

Secondly, they have improved insolubility in water at 37° C. compared to other forms, including an amorphous form.

Thirdly, the L-lysine (1:2) adduct described herein is unexpectedly more soluble in a saline solution compared to other forms, including an amorphous form.

### Compound of Formula I

As noted above, a compound of formula I—also called QTX125 herein—is 3-(3-Furyl)-N-{4-[(hydroxyamino)carbonyl]benzyl}-5-(4-hydroxy phenyl)-1H-pyrrole-2-carboxamide and has the following chemical formula:

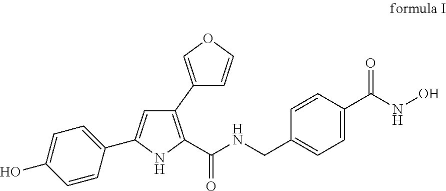 [SMILES: O=C(NO)c1ccc(CNC(=O)c2[nH]c(-c3ccc(O)cc3)cc2-c2ccoc2)cc1]

Methods of preparing a compound of formula I, and evidence of its biological activity for application in various medical treatments, are described in e.g. WO 2018/087082, the contents of which are incorporated herein by reference. A compound of formula I can be used directly in the preparation of the crystalline forms of the present invention, or an adduct may be formed first (see below) before the crystalline form is prepared.

A crystalline form of a compound of formula I according to the present invention has a characteristic PXRD pattern having peaks at 2θ=20.4°, 21.8°, 22.0°, 22.7°, and 23.9° (±0.3° 2θ).

The peaks of the PXRD pattern may further be described in terms of the relative intensities of the peaks. In the following, relative intensity is calculated as a percentage of the highest intensity peak as follows: relative intensity (%)=[peak intensity/intensity of highest intensity peak]×100. The peak intensity values are provided in counts. In this application, we use vs=very strong (relative intensity >70% of highest peak); s=strong (45% 5 relative intensity <70%); m=medium (20% 5 relative intensity <45%); w=weak (5% 5 relative intensity <20%) and vw=very weak (relative intensity <5%).

In some embodiments, such as when the crystalline form is Form 2, the above-mentioned peaks have the following relative intensity profile: 2θ (±0.3° 2θ)=20.4° (w); 21.8° (vs); 22.0° (s); 22.7° (w); and 23.9° (m).

In some embodiments, the characteristic PXRD pattern further has at least one peak selected from 2θ=9.0°, 12.6°, 26.6°, 30.5°, 32.2° (±0.3° 2e).

Preferably, the characteristic PXRD pattern has at least two, more preferably at least three, more preferably at least four, and most preferably all of the above-mentioned further peaks.

For example, the characteristic PXRD pattern may further have the following combinations of peaks:
2θ (±0.3° 29)=9.0° and 12.6°, or 9.0° and 26.6°, or 9.0° and 30.5°, or 9.0° and 32.2°, or 12.6° and 26.6°, or 12.6° and 30.5°, or 12.6° and 32.2°, or 26.60 and 30.5°, or 26.60 and 32.2°, or 30.5° and 32.2°; or
2θ (±0.3° 29)=9.0° and 12.6° and 26.6°, or 9.0° and 12.6° and 30.5°, or 9.0° and 12.6° and 32.2°, or 9.0° and 26.6° and 30.5°, or 9.0° and 26.6° and 32.2°, or 9.0° and 30.5° and 32.2°, or 12.6° and 26.6° and 30.5°, or 12.6° and 26.6° and 32.2°, or 12.6° and 30.5° and 32.2°, or 26.6° and 30.5° and 32.2°; or
2θ (±0.3° 29)=9.0° and 12.6° and 26.6° and 30.5°, or 9.0° and 12.6° and 26.6° and 32.2°, or 9.0° and 12.6° and 30.5° and 32.2°, or 12.6° and 26.6° and 30.5° and 32.2°; or
2θ (±0.3° 29)=9.0° and 12.6° and 26.6° and 30.5° and 32.2°.

In preferred embodiments, the characteristic PXRD pattern includes a peak at 2θ=26.6° (±0.3° 2e).

In some embodiments, the above-mentioned peaks have the following relative intensity profile: 2θ (±0.3° 2θ)=9.0° (m), 12.6° (vw), 26.6° (m), 30.5° (w), and 32.2° (w).

Further preferably, the characteristic PXRD pattern of a crystalline form of a compound of formula I according to the present invention has the peaks mentioned in Table A below:

TABLE A
2θ (degree), ±0.3° 2θ

9.0
12.6
13.0
14.2
16.5
16.9
20.4
21.8
22.0
22.7
23.9
26.6
30.5
32.2

Further preferably, the peaks of Table A have the following relative intensity profile: 2θ (±0.3° 2θ)=9.0° (m), 12.6° (vw), 13.0° (w), 14.2° (m), 16.5° (m), 16.9° (m), 20.4° (w), 21.8° (vs), 22.0° (s), 22.7° (w), 23.9° (m), 26.6° (m), 30.5° (w), and 32.2° (w).

In addition to the above-mentioned peaks, the characteristic PXRD pattern of a crystalline form of a compound of formula I according to the present invention may further have one or more, such as two or three or all of, the following peaks, with preferable relative intensity profiles mentioned alongside in parentheses: 2θ (±0.3° 2θ)=8.4° (w), 10.3° (w), 15.8° (m), 18.8° (s), 20.9° (w), 21.2° (m), 23.2° (w), 23.4° (w), and/or 29.1° (w).

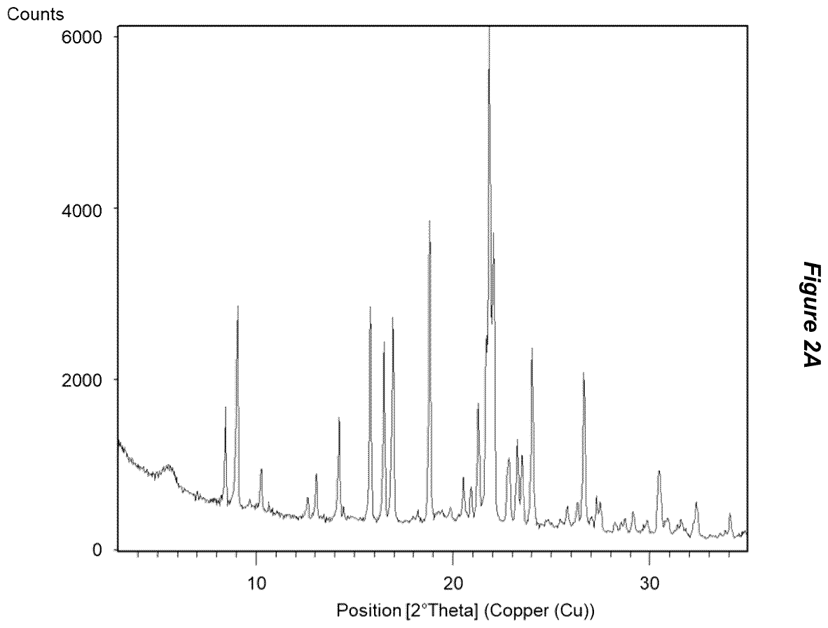

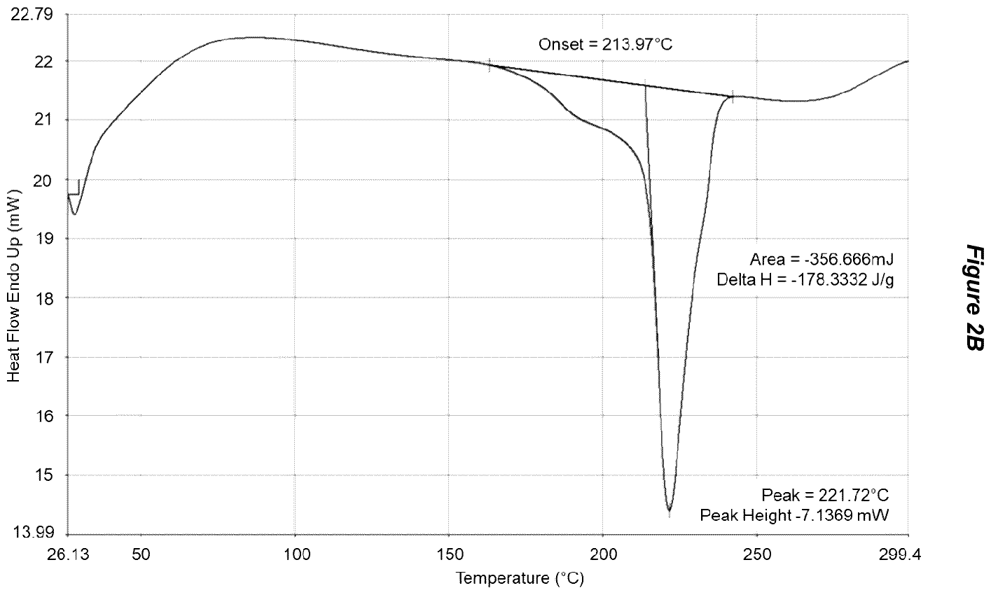

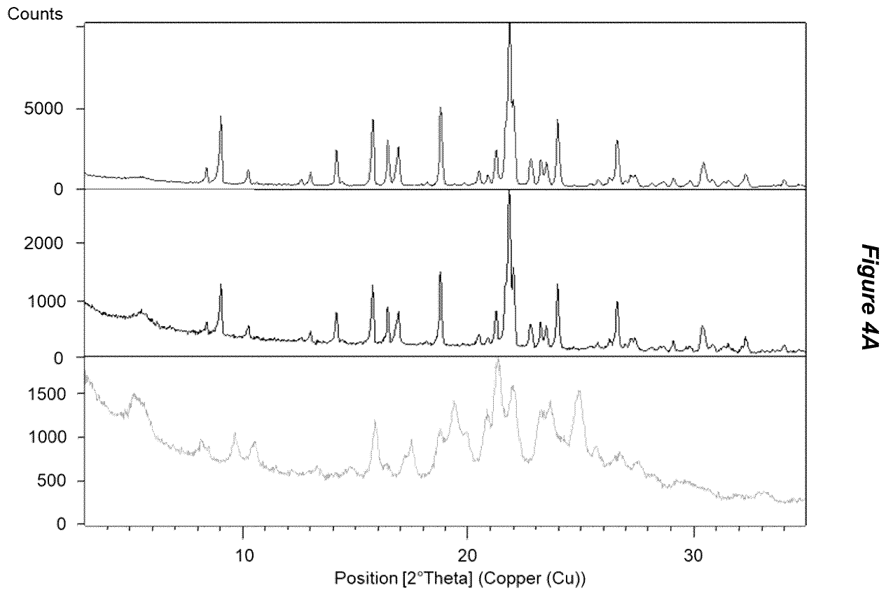

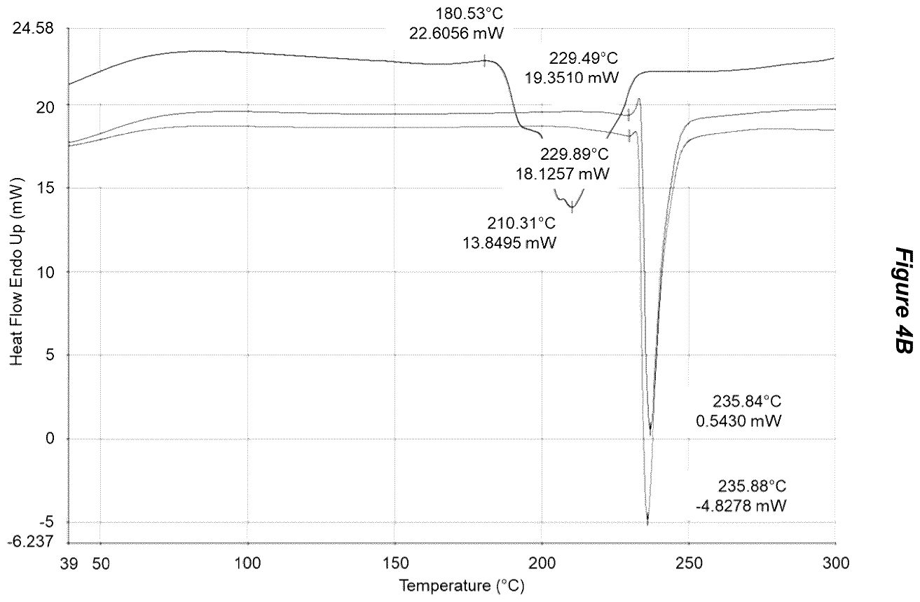

In preferred embodiments, the PXRD pattern is substantially similar to, or the same as, the PXRD pattern shown in FIG. 2A or FIG. 3A or FIG. 4A uppermost or middle, or FIG. 5A.

A crystalline form of a compound of formula I having a PXRD pattern that is substantially similar to, or the same as, the PXRD pattern shown in FIG. 2A or FIG. 3A or FIG. 4A uppermost or middle, or FIG. 5A may be referred to herein as Form 2. That is, the Form 2 crystalline polymorph of a compound of formula I as referred to herein has the characteristics described above.

The PXRD pattern may be measured on any suitable diffractometer. As an example, the PXRD patterns of the present application were obtained using a PANalytical X'Pert PRO diffractometer with a PixCEL detector. Suitable diffractometers are typically used in transmission geometry. Suitable diffractometers use Cu Kα radiation, for example at 1.54056 Å, and may operate at 40 kV and 40 mA. A measurement range may be 2-38° 2θ.

Analysis may be performed by any suitable means, such as with appropriate software. Any suitable sample preparation method may be used.

A crystalline form of a compound of formula I as detailed herein preferably has a purity of at least 97%. More preferably, a crystalline form of a compound of formula I as described herein has a purity of at least 97.5%, more preferably, 98%, more preferably 98.5%, and most preferably 99% or higher such as 99.5%. The purity described herein is as measured by high-performance liquid chromatography (HPLC). A particularly suitable method is provided in the examples.

A DSC profile of a crystalline form of a compound of formula I as detailed herein further preferably shows an exothermic peak at between 220-225° C., further preferably between 221 and 223° C. Most preferably, the DSC profile of a crystalline form of a compound of formula I as detailed herein is substantially similar to, or the same as, that shown in FIG. 2B.

Adducts of Compound of formula I

In the invention as it refers to adducts, the compound of formula I is adducted with at least one other molecule. Typically, this adduction occurs before crystallization. For example, in the exemplified adduct of QTX125 and L-lysine described herein, the L-lysine and QTX125 are mixed before crystallization occurs.

In some embodiments, the adduct is an adduct with an amino acid, such as a natural amino acid. Preferably, the adduct is an adduct with lysine, most preferably L-lysine.

Preferably, the adduct is a (1:2) adduct so that in the crystalline form there are two molecules of the other molecule for every one molecule of the compound of formula I.

In particularly preferred and exemplified embodiments, the adduct is a (1:2) adduct of the compound of formula I with L-lysine i.e. there are two molecules of L-lysine for every molecule of the compound of formula I. This adduct may be prepared by a method substantially as described herein.

A crystalline form of an adduct of a compound of formula I according to the present invention has a characteristic PXRD pattern having peaks at 2θ=20.4, 21 0.8°, 22.0°, 22.7° and 23.9°, (±0.3° 2θ). The meaning of “±0.3° 2θ” is as given above in relation to the crystalline forms of the compound of formula I.

In some embodiments, such as when the crystalline form is a crystalline form of a 1:2 adduct of the compound of formula I with L-lysine, the above-mentioned peaks have the following relative intensity profile: 2θ (±0.3° 2θ)=20.4° (m); 21.8° (m); 22.0° (vs); 22.7° (vs); and 23.9° (vs). The definitions of relative intensity are as given above in relation to the crystalline forms of the compound of formula I.

In some embodiments, the above-mentioned peaks of the crystalline form of a 1:2 adduct of the compound of formula I with L-lysine are characteristic at 2θ=20.6, 21 0.8°, 22.3°, 22.7° and 23.7° (±0.1° 2θ).

In some embodiments, the characteristic PXRD pattern further has at least one peak selected from 2θ=11.2°, 11.7°, 15.1°, 18.0°, and 26.1° (±0.3° 2e). Preferably, the characteristic PXRD pattern has at least two, more preferably at least three, more preferably at least four, and most preferably all of the above-mentioned further peaks.

For example, the characteristic PXRD pattern may further have the following combinations of peaks:
2θ (±0.3° 29)=11.2° and 11.7°, or 11.2° and 15.1°, or 11.2° and 18.0°, or 11.2° and 26.1°, or 11.7° and 15.1°, or 11.7° and 18.0°, or 11.7 and 26.1°, or 15.1° and 18.0°, or 15.1° and 26.1°, or 18.0° and 26.1°; or
2θ (±0.3° 29)=11.2° and 11.7° and 15.1°, or 11.2° and 11.7° and 18.0°, or 11.2° and 11.7° and 26.1°, or 11.7° and 15.1° and 18.0°, or 11.7° and 15.1° and 26.1°, or 11.7° and 18.0° and 26.1°, or 15.1° and 18.0° and 26.1°; or
2θ (±0.3° 29)=11.2° and 11.7° and 15.1° and 18.0°, or 11.2° and 11.7° and 15.1° and 26.1°, or 11.2 and 11.7° and 18.0° and 26.1°, or 11.7° and 15.1° and 18.0° and 26.1°; or
2θ (±0.3° 29)=11.2° and 11.7° and 15.1° and 18.0° and 26.1°.

In some embodiments, the characteristic PXRD pattern includes a peak at 2θ=26.1° (±0.3° 2θ).

In some embodiments, the above-mentioned peaks have the following relative intensity profile: 2θ (±0.3° 2θ)=11.2° (m), 11.7° (w), 15.1° (w), 18.0° (m), 26.1° (m).

Further preferably, the characteristic PXRD pattern of a crystalline form of a compound of formula I according to the present invention has the peaks mentioned in Table B below:

TABLE B
2θ (degree), ±0.3° 2θ

5.6
8.2
11.2
11.7
13.0
15.1
16.9
17.7
18.0
20.4
21.8
22.0
22.7
23.9
26.1

In some embodiments, the peaks of Table B have the following relative intensity profile: 20 (±0.3° 2θ)=5.6° (s), 8.2° (vs), 11.2° (m), 11.7° (w), 13.0° (w), 16.9° (w), 17.7° (m), 18.0° (m), 20.4° (m), 21.8° (w), 22.0° (m), 22.7° (s), 23.9° (s), 26.1° (m).

In addition to the above-mentioned peaks, the characteristic PXRD pattern of a crystalline form of an adduct of a compound of formula I according to the present invention may further have one or more, such as two or three or all of, the following peaks, with preferable relative intensity profiles mentioned alongside in parentheses: 2θ (±0.3° 2θ)=9.7° (w), 10.8° (w), 15.8° (w), 18.8° (s), 23.2° (s), 23.4° (s) and 24.8° (m).

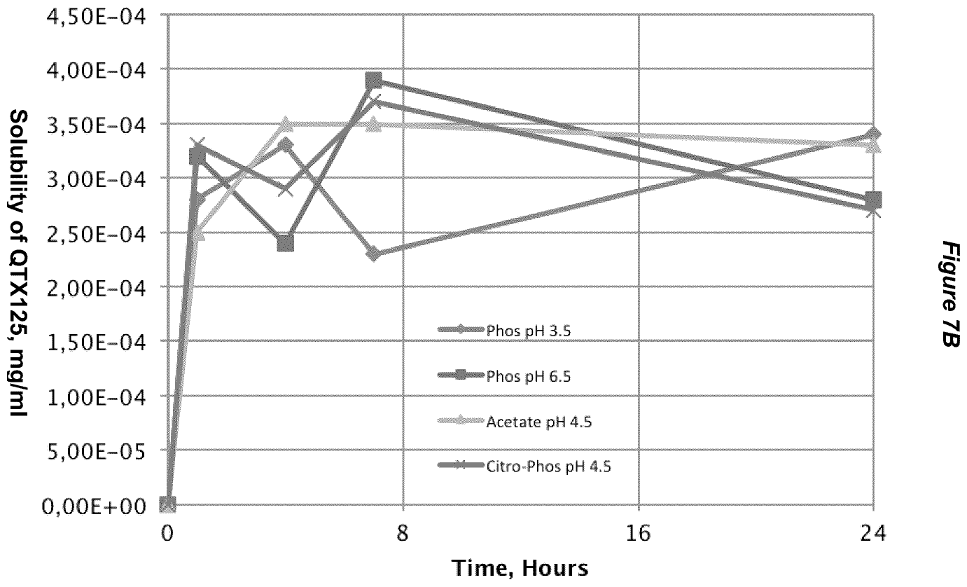

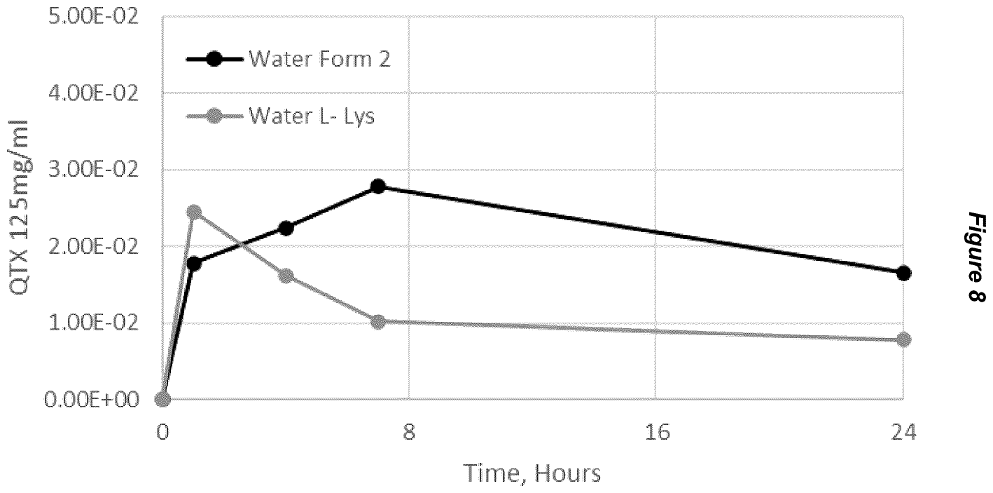

In preferred embodiments, the PXRD pattern is substantially similar to, or the same as, the PXRD pattern shown in FIG. 9.

A crystalline form of an adduct of a compound of formula I as detailed herein preferably has a purity of at least 97%. More preferably, a crystalline form of an adduct of a compound of formula I as described herein has a purity of at least 97.5%, more preferably, 98%, more preferably 98.5%, and most preferably 99% or higher such as 99.5%. As discussed elsewhere, the method of measurement of the purity typically uses HPLC.

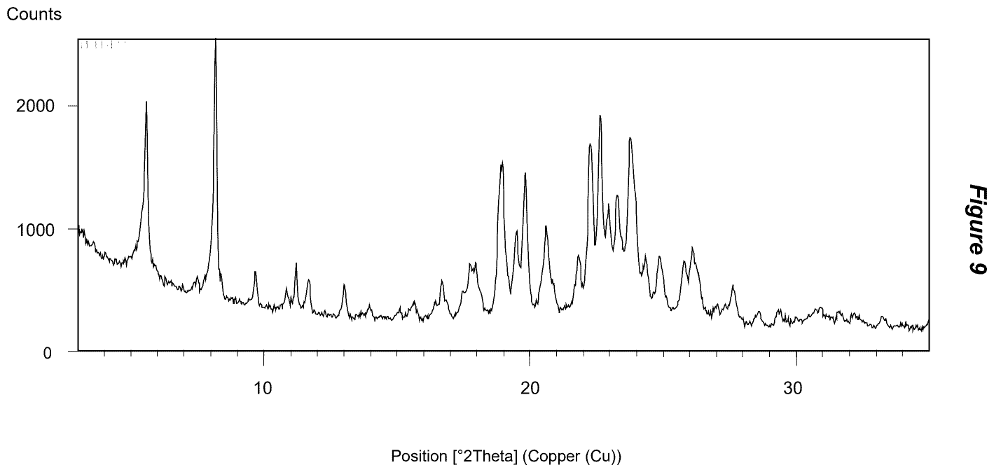

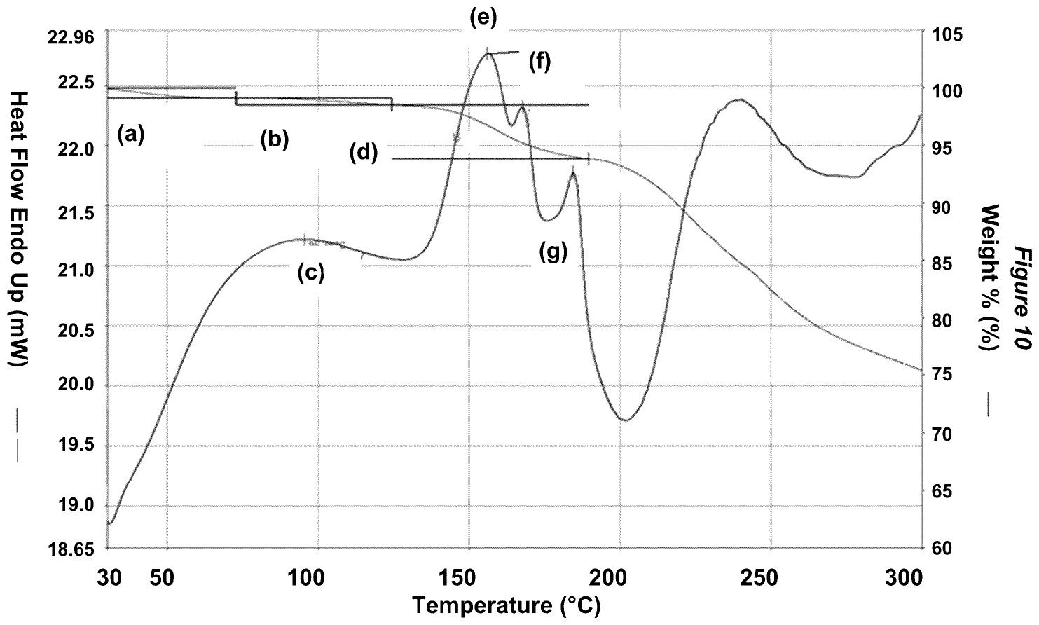

A DSC profile of a crystalline form of an adduct of a compound of formula I as detailed herein further preferably shows an exothermic peak at between 150-160° C., more preferably between 153 and 157° C.; and/or an exothermic peak at between 162-170° C., more preferably between 165 and 169° C.; and/or and exothermic peak at between 180-190° C., more preferably between 182 and 186° C. Most preferably, the DSC profile of a crystalline form of a compound of formula I as detailed herein is substantially similar to, or the same as, that shown in FIG. 10.

### Pharmaceutical Compositions

A pharmaceutical composition according to the present invention comprises a crystalline form of a compound of formula I or an adduct thereof according to the present invention.

A pharmaceutical composition according to the present invention comprises at least one crystalline form of a compound of formula I or an adduct thereof as described herein. In some embodiments, two crystalline forms of a compound of formula I or an adduct thereof may be present in the pharmaceutical composition. For example, a pharmaceutical composition according to the present invention may comprise a crystalline form of a compound of formula I which is Form 2, as well as a crystalline form of an adduct of a compound of formula I such as a 1:2 adduct with lysine.

In some embodiments, the crystalline form of the compound of formula I or adduct thereof in the pharmaceutical composition is in particulate form. In such cases, the pharmaceutical composition can be described as a suspension or a slurry. Put differently, the compound of formula I or adduct thereof is solid when the pharmaceutical composition is used.

The particles or crystallites making up such a pharmaceutical composition may have any suitable average particle size, and the invention is not limited thereby. By way of example, the average particle size may be at least 10 μm, at least 15 μm or at least 25 μm. By way of example, the average particle size may be up to 100 μm, up to 90 μm or up to 80 μm. Combinations of these values may be used to provide example average particle size ranges. Other exemplary average particle size ranges may be from 10 to 100 μm, such as from 15 to 80 μm or from 25 to 60 μm. In such cases, the average particle size refers to a mean particle size, taking the longest length of the particle. The sample size taken for the measurement of average particle size may be any appropriate, such as 5 particles or 10 particles or 30 particles or 50 particles or more. Suitable measurement methods may include optical microscopy, or scanning electron microscopy for example, and particularly optical microscopy.

The particles or crystallites may have any suitable shape and the subject application is not limited thereby. Exemplary shapes include spherical, cuboid, pyramidal or rod-like.

Exemplary final concentrations of the QTX125 in pharmaceutical compositions according to the invention are at least 8 mg/mL, optionally up to 20 mg/mL, such as 8.5 mg/mL or more, 9 mg/mL or more and more preferably 9.5 mg/mL or more.

In some embodiments, the crystalline form of the compound of formula I or adduct thereof according to the present invention is used to prepare liquid pharmaceutical compositions. In such cases, the crystalline form of the compound of formula I or adduct thereof is dissolved (in suitable medium) to provide the pharmaceutical composition. In such cases, the compound of formula I is not solid when used.

A pharmaceutical composition according to the present invention may comprise, in addition to the crystalline form of the compound of formula I or adduct thereof as described herein, one or more other pharmaceutically acceptable ingredients well known to those skilled in the art, including, but not limited to, pharmaceutically acceptable carriers, diluents, excipients, adjuvants, buffers, pH modifiers, preservatives, anti-oxidants, bacteriostats, stabilisers, suspending agents, solubilisers, surfactants (e.g., wetting agents), colouring agents, and isotonicizing solutes (i.e., which render the formulation isotonic with the blood, or other relevant bodily fluid, of the intended recipient). Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 18th edition, Mack Publishing Company, Easton, Pa., 1990; and Handbook of Pharmaceutical Excipients, 2nd edition, 1994.

Optionally, the pharmaceutical composition according to the present invention further comprises a buffer (i.e. the composition further comprises buffer salts dissolved therein). Optionally, the said buffer may be selected from the group of MES, Bis-Tris, ADA, ACES, PIPES, MOPSO, BES, MOPS, TES, HEPES, DIPSO, MOBS, TAPSO, Tris-HCl, HEPPSO, POPSO, TEA, EPPS, Tricine, Gly-Gly, Bicine, HEPBS, TAPS, AMPD, TABS, AMPSO, CHES, CAPSO, APS, CHAPS, CABS, Phosphate and histidine or a combination of the above.

Without wishing to be bound by theory, it is believed that the use of a buffer may help to stabilise the composition at physiological pH.

The concentration of the buffer salt in the aqueous pharmaceutical composition may range from 1 mM to 1 M, preferably 1 mM to 100 mM, preferably 5 mM to 50 mM, preferably 5 mM to 20 mM.

The pharmaceutical composition may also comprise counter-ions and salts, such as sodium counter ions, chloride ions or NaCl dissolved is solution.

The pharmaceutical composition may also comprise, in addition to the active ingredient which is the crystalline form of a compound of formula I or an adduct thereof as described herein, one or more other active agents, for example, one or more other therapeutic or prophylactic agents.

In some embodiments, a crystalline form of a compound of formula I or adduct thereof according to the present invention, or pharmaceutical composition according to the present invention, can be used with at least one other drug to provide a combination therapy. This other drug or drugs may be part of the same composition or may be provided as a separate composition and can be administered at the same time or at different times.

Optionally, a pharmaceutical composition of the present invention comprises:
water
optionally salts, such as buffer salts or dissolved NaCl;
a crystalline form of a compound of formula I or an adduct thereof as described herein; and
wherein the pH of the pharmaceutical formulation is between pH 7 and pH 8.

In some embodiments, a crystalline form of a compound of formula I or adduct thereof as described herein can be used to prepare an aqueous pharmaceutical formulation for injection, or a dry pharmaceutical formulation obtainable by drying such aqueous pharmaceutical formulation. Such a pharmaceutical formulation may be prepared from a crystalline form of a compound of formula I or adduct thereof and a compound of formula II:

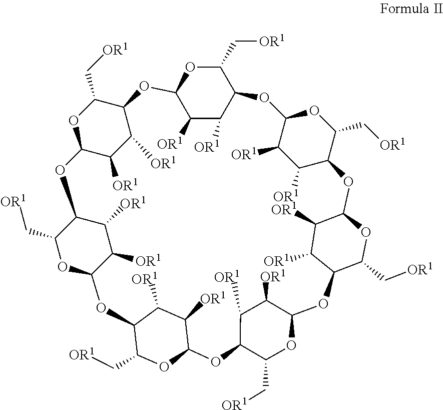

wherein each R¹ is independently selected from the group of: —H or

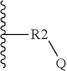

wherein R² is either absent or is a C₁₋₄alkyl;
Q is selected from the group of: —H, —SO₃, —OH, —C(O)R³ or —C(OH)R³₂; and
R³ is independently selected from —H or C₁₋₄alkyl;
wherein the molar ratio of the compound of formula I to the compound of formula II is from 1:50-1:2; and
wherein the pH of the pharmaceutical formulation is between pH 7 and pH 8.

In some embodiments, the compound of formula II is selected from the group of: β-cyclodextrin, (C₁₋₄alkyl)-β-cyclodextrin, (hydroxy-C₁₋₄alkyl)-β-cyclodextrin and sulfobutyl ethers of β-cyclodextrin, such as hydroxy propyl β cyclodextrin or sulfobutyl ether β cyclodextrin (SBβCD).

A skilled person can determine the desired concentrations or amounts of the components of the active ingredients in such a formulation. Exemplary final concentrations of the compound of formula I are at least 8 mg/mL, optionally up to 20 mg/mL, such as 8.5 mg/mL or more, 9 mg/mL or more and mor preferably 9.5 mg/mL or more. Exemplary molar ratios of the compound of formula I to the compound of formula II are from 1:40 to 1:2.5, preferably from 1:30 to 1:2.5, preferably from 1:25 to 1:2.5, preferably from 1:20 to 1:2.5, such as from 1:15 to 1:2.5, preferably from 1:10 to 1:2.5, preferably from 1:9 to 1:2.5, preferably from 1:8 to 1:2.5, preferably from 1:6 to 1:2.5, more preferably from 1:4.5 to 1:2.5.

Optionally, the pharmaceutical composition according to the present invention is substantially free of meglumine.

### Preparation Methods

A crystalline form of a compound of formula I may be prepared by a method comprising the steps of:
(i) adding a compound of formula I to water to form a suspension;
(ii) heating the suspension;
(iii) adding one or more organic solvents before cooling; and
(iv) isolating a crystalline form of a compound of formula I or an adduct thereof.

In preferred embodiments, the one or more organic solvents comprise one or more of a C₁₋₅ alcohol, tetrahydrofuran (THF) and dioxane. More preferably, the one or more organic solvents comprise, and most preferably consist of, one or more of propanol, ethanol, THF and dioxane, and most preferably include all of these. Preferably, the volume ratios of C₁₋₅ alcohol:THF:dioxane are up to 12.5:10:1.5, such as 6:3:0.867.

Preferably, heating is carried out to a temperature of 70-120° C., such as 90-110° C.

Preferably, step (iii) occurs with agitation. Agitation may be provided by any suitable means. Preferably, agitation occurs for several hours between steps (iii) and (iv).

In some embodiments, after cooling at step (iii), step (iv) comprises isolating the solid by filtration, treating with solvents by displacement, and drying in vacuo with heating. In some embodiments, the solvents include ethanol and water. In some embodiments, the step of treating with solvents includes treating with ethanol, then water, then ethanol. In some embodiments, the ethanol and water are used in an amount of between 1-3 vol (such as 2 vol) i.e. 1-3 ml per 1 g of the crystalline form. In some embodiments, drying in vacuo with heating includes heating to up to 100° C., such as up to 80° C. or up to 70° C. In some embodiments, the heating is up to at least 30° C., such as up to 35° C. or up to 40° C. Combinations of any of those end-points may be used to provide a suitable range. In some embodiments, the heating is between 30-100° C., such as between 40-75° C., such as between 40-60° C.

A crystalline form of an adduct of a compound of formula I may be prepared by a method comprising the steps of:
(i) adding a compound to be adducted to ethanol to form a first mixture;
(ii) adding a compound of formula I to water and one or more organic solvents to form a second mixture;
(iii) combining the first and second mixtures to form a composition;
(iv) cooling the composition; and
(v) isolating a crystalline form of an adduct of a compound of formula I.

In preferred embodiments, the compound to be adducted is an amino acid, preferably an L-amino acid, preferably lysine, and most preferably L-lysine. Preferably, the adduct is as discussed above for the crystalline form of the adduct of a compound of formula I.

When a 1:1 adduct is desired, the compound to be adducted and the compound of formula I should be present as 1:1 equivalents. When a 1:2 adduct is desired, the compound to be adducted and the compound of formula I should be present as 2:1 equivalents, respectively. Corresponding adducts should be provided in corresponding equivalent amounts.

In preferred embodiments, the one or more organic solvents comprises, more preferably is, tetrahydrofuran (THF).

In preferred embodiments, the addition and combining steps (i) to (iii) occur at 55-65° C.

In preferred embodiments, the cooling step comprises two cooling steps (iii)-a and (iii)-b. In a first cooling step (iii)-a, the composition is cooled for a relatively short time, such as 0.3-1 hours, and the temperature is reduced by around 5-15° C. In a second cooling step (iii)-b, the composition is cooled for a relatively long time, such as several hours, e.g. 2-24 hours, such as 10-20 hours, to room temperature.

In preferred embodiments, steps (i) to (iv) occur with agitation. Agitation may be performed by any suitable means, such as stirring. The stirring device used is not particularly limited, suitable stirring devices may include a vortex mixer, a magnetic stirrer, a helix mixer or a paddle type stirrer.

### Medical Use, Methods of Treatment

In a further aspect, the present invention relates to a crystalline form of a compound of formula I or an adduct thereof according to the present invention, or a pharmaceutical composition comprising the crystalline form of a compound of formula I or an adduct thereof, for use in the manufacture of a medicament.

The present invention also relates to a crystalline form of a compound of formula I or an adduct thereof according to the present invention, or a pharmaceutical composition comprising the crystalline form of a compound of formula I or an adduct thereof, for use in the manufacture of a medicament for the treatment of cancer.

Alternatively, the present invention relates to a crystalline form of a compound of formula I or an adduct thereof according to the present invention, or a pharmaceutical composition comprising the crystalline form of a compound of formula I or an adduct thereof, for use in the manufacture of a medicament for the treatment of an autoimmune disease.

In a further aspect, the present invention relates to a crystalline form of a compound of formula I or an adduct thereof according to the present invention, or a pharmaceutical composition comprising the crystalline form of a compound of formula I or an adduct thereof for use as a medicament.

Preferably, the present invention relates to a crystalline form of a compound of formula I or an adduct thereof according to the present invention, or a pharmaceutical composition comprising the crystalline form of a compound of formula I or an adduct thereof, for use in the treatment of cancer.

Alternatively, the present invention relates to a crystalline form of a compound of formula I or an adduct thereof according to the present invention, or a pharmaceutical composition comprising the crystalline form of a compound of formula I or an adduct thereof, for use in the treatment of an autoimmune disease.

In a further aspect, the present invention relates to a method of treatment comprising administering a pharmaceutical composition comprising a crystalline form of a compound of formula I or an adduct thereof according to the present invention, or a pharmaceutical composition comprising the crystalline form of a compound of formula I or an adduct thereof, to a patient in need of such treatment.

Preferably, the present invention relates to a method of treating cancer comprising administering a crystalline form of a compound of formula I or an adduct thereof according to the present invention, or a pharmaceutical composition comprising the crystalline form of a compound of formula I or an adduct thereof, to a patient in need of such treatment.

Alternatively, the present invention relates to a method of treating an autoimmune disease comprising administering a crystalline form of a compound of formula I or an adduct thereof according to the present invention, or a pharmaceutical composition comprising the crystalline form of a compound of formula I or an adduct thereof, to a patient in need of such treatment.

Preferably, the cancer is selected from breast cancer, chronic myelogenous (or myeloid) leukaemia (CML), colorectal cancer, lymphoma (such as non-Hodgkin lymphoma), fibrosarcoma, gastric cancer, glioblastoma, kidney cancer, liver cancer, lung cancer, melanoma, nasopharyngeal cancer, oral cancer, orthotopic multiple myeloma, osteosarcoma, ovarian cancer, pancreatic cancer, and prostate cancer.

Preferably, the autoimmune disease is selected from autoimmune hepatitis; an inflammatory demyelinating disease of the central nervous system; systemic lupus erythematosus; acute anterior uveitis; Sjogren's syndrome; rheumatoid arthritis; diabetes mellitus type 1; Graves' disease; and inflammatory bowel disease.

An inflammatory demyelinating disease of the central nervous system is a disease wherein myelin-supporting cells of the central nervous system, such as oligodendrocytes, and/or the myelin lamellae are destroyed. Demyelination leads to a disruption in neural signals between the brain and other parts of the body, ultimately resulting in a range of signs and symptoms, including physical, mental, and sometimes psychiatric problems.

Specific, non-limiting examples of inflammatory demyelinating diseases are multiple sclerosis (MS), including relapsing-onset MS, progressive-onset MS, optic-spinal MS; neuromyelitis optica; acute-disseminated encephalomyelitis; acute haemorrhagic leukoencephalitis; Balo concentric sclerosis; Schilder's disease; Marburg MS; tumefactive MS; solitary sclerosis; optic neuritis; transverse myelitis; Susac's syndrome; leukoaraiosis; myalgic encephalomyelitis; Guillain-Barre syndrome; progressive inflammatory neuropathy; leukodystrophy, including adrenoleukodystrophy and adrenomyeloneuropathy. Preferably, the autoimmune disease is multiple sclerosis or acute-disseminated encephalomyelitis.

More particularly it is acute-disseminated encephalomyelitis, or more particularly and most preferably it is multiple sclerosis.

Preferably, the autoimmune disease is selected from autoimmune hepatitis and an inflammatory demyelinating disease of the central nervous system.

In a particularly preferred embodiment, the autoimmune disease is an inflammatory demyelinating disease of the central nervous system as described above.

In another particularly preferred embodiment, the autoimmune disease is autoimmune hepatitis.

The present inventors have found that QTX125, unlike other histone deacetylase inhibitors, advantageously show no evidence of genotoxicity, in particular of clastogenicity or aneugenicity. Similarly, it has unexpectedly been observed that QTX125 possess improved pharmacokinetic properties, in particular higher half-lives and distribution volumes, than other histone deacetylase inhibitors.

### Administration

Preferably, a crystalline form of a compound of formula I or an adduct thereof according to the present invention, or a pharmaceutical composition comprising the crystalline form of a compound of formula I or an adduct thereof, is administered via injection. Administration may be both via infusion (continuous) or bolus (discreate) administration.

The method of administration via injection may be, for example, subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal injection.

Preferably, the administration is by intravenous infusion or intravenous injection (bolus administration). More preferably, the administration is by intravenous infusion.

### Subject/Dosing

The subject for administration may be any animal. Preferably, the subject is a mammal, such as a rat, mouse, feline, canine, equine, porcine, ovine, bovine, primate or human. Preferably, the subject is a human patient.

In general, the effective amount of the compound of formula I to be administered will depended on a range of factors, such as the severity of the disorder being treated and the subject's weight. The active compounds will normally be administered one or more times a day for example 1, 2, 3, or 4 times daily, with typical total daily doses in the range from 0.01 up to 1,000 mg/kg/day.

Preferably, the compound of formula I is administered to human patients at a dosage of 0.5 to 50 mg/kg, preferably from 0.5 to 30 mg/kg, preferably from 1 to 20 mg/kg, more preferably from 5 to 10 mg/kg.

Preferably, the compound of formula I is administered to human patients at a dosage of from 25 mg to 4500 mg, preferably from 50 mg to 3000 mg, preferably from 250 mg to 1500 mg per day.

The compounds of the present invention can be used with at least one other drug to provide a combination therapy. This other drug or drugs may be part of the same composition, or may be provided as a separate composition and can be administered at the same time or at different times.

### Kits

Another aspect of the invention relates to a kit comprising a crystalline form of a compound of formula I or an adduct thereof according to the present invention. In addition, the kit comprises a pharmaceutically acceptable grade of water, buffer solution or saline solution for use in preparing a dosage form. In some embodiments, the crystalline form of the compound of formula I or adduct thereof of the present invention is provided in a separate container to the pharmaceutically acceptable grade of water, buffer solution or saline solution in the kit.

Preferably, the crystalline form of the compound of formula I or an adduct thereof is provided in a suitable container and/or with suitable packaging.

The kit may also include one or more delivery systems for delivering or administering the components provided therein e.g. a syringe and needle. The kit may also include directions for use (e.g. instructions for treating a subject).

Preferably, the kit also includes instructions for use, e.g. written instructions on how to administer the composition (e.g. the injection procedure). Most preferably, the kit includes written instruction on how to prepare a suitable pharmaceutical composition from the components provided, and how to subsequently administer the prepared pharmaceutical composition.

As will be appreciated by one of skill in the art, features and preferred embodiments of one aspect of the invention will also pertain to other aspects of the invention.

### EXAMPLES

The following non-limiting examples are provided to illustrate the invention.

### Example 1—Characterisation of Crude QTX125 and Form 2

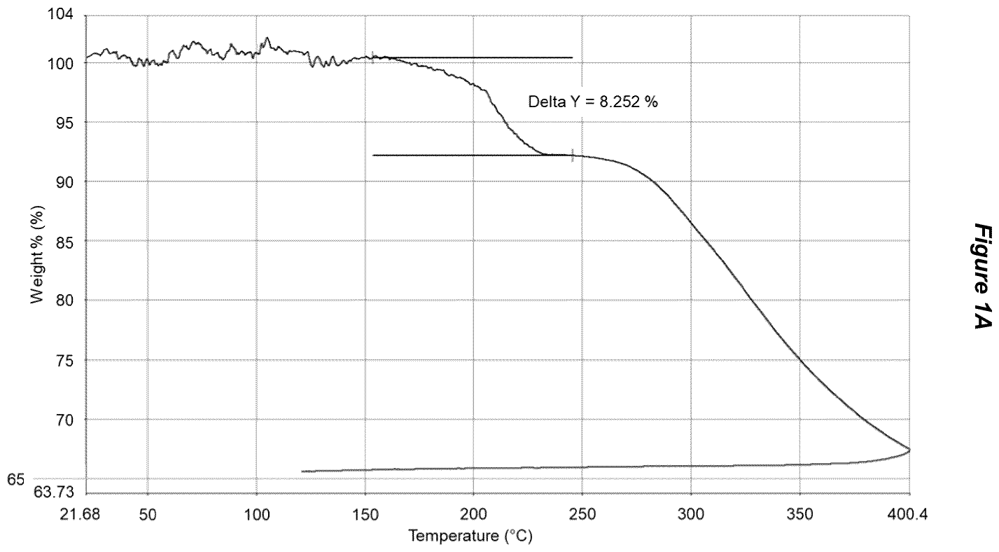

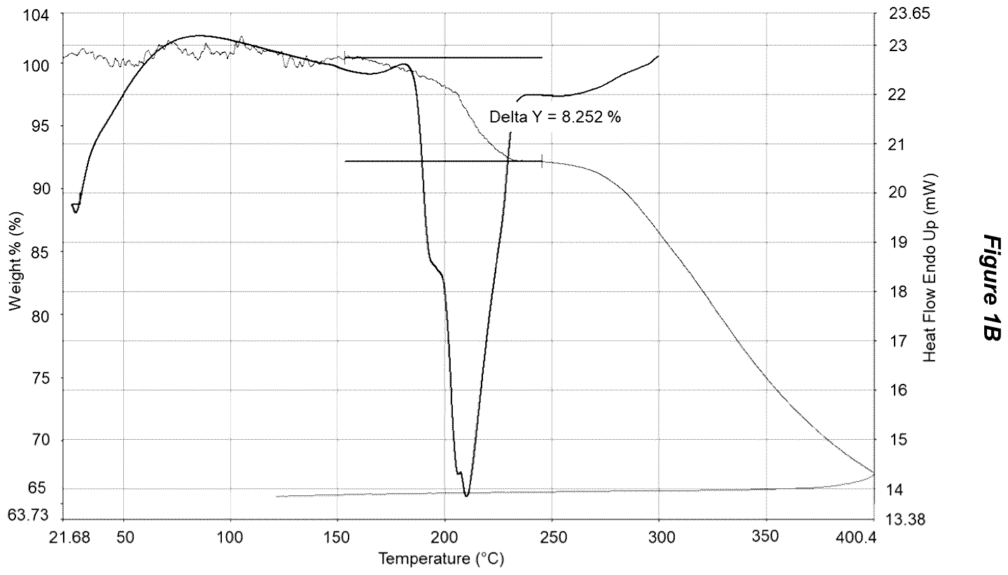

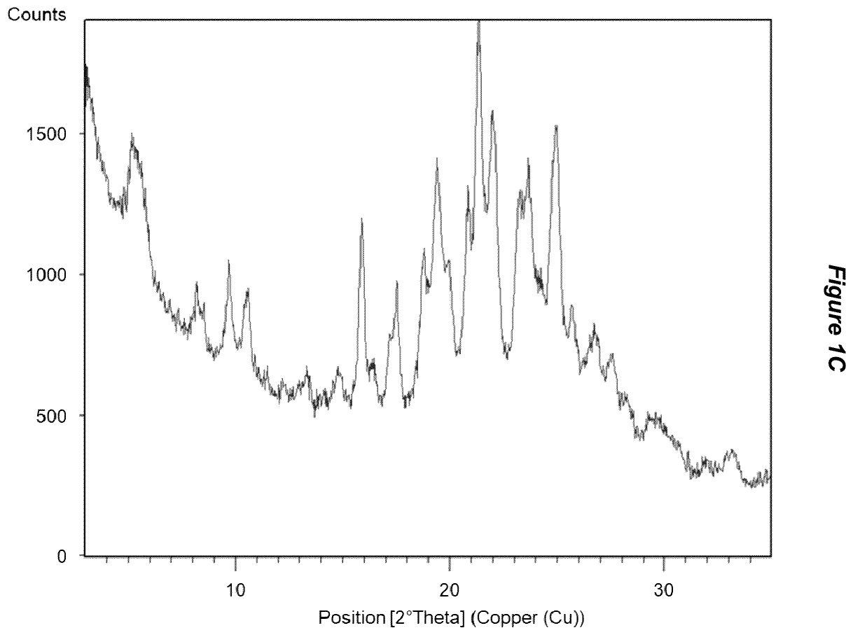

A scaled-up process was used to obtain QTX125 and initial characterisation and purity assessment of crude QTX125 was performed. FIG. 1A shows a non-solvated substrate that decomposes post 150° C., which the inventors theorise is most likely via hydroxylamine release ahead of total decomposition. FIG. 1B shows the DSC and TGA overlay, whereby no formal endothermic melt is noted. Instead a large exothermic event coincides with the onset of decomposition as judged by TGA. A minor phase transition or melt is noted at approximately 150° C. and again at 180° C. FIG. 1C shows the PXRD profile of crude QTX125. The peaks identified are relatively broad, and an amorphous halo effect is apparent. The purity of crude QTX125 was measured to be 94.30% by high-performance liquid chromatography (HPLC). [The HPLC method is provided elsewhere.] Four key impurities were identified, as shown in Table 1.

TABLE 1
Identification of impurities in crude QTX125

| RRT | 0.19 | 0.89 | 1.03 | 1.12 |
|---|---|---|---|---|
| % | 0.78 | 0.53 | 0.78 | 1.66 |

Herein, RRT stands for relative retention time. Relative retention time. The relative retention time was calculated using the following formula: RRT=(Tᵢₘₚᵤᵣᵢₜᵢₑₛ/T_{reference}) where T=retention time, the reference peak was the peak of QTX125.

The inventors believe that the RRT at 1.03 corresponds with the carboxamide of QTX125 i.e.

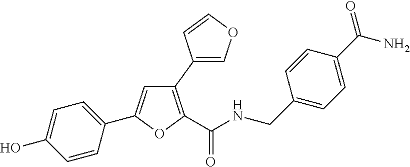 [SMILES: NC(=O)c1ccc(CNC(=O)c2oc(-c3ccc(O)cc3)cc2-c2ccoc2)cc1]

and that the RRT at 1.12 corresponds to the carboxylic acid of QTX125 i.e.

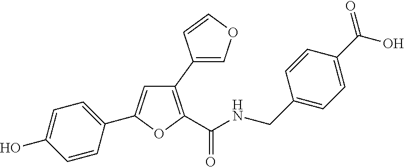 [SMILES: O=C(O)c1ccc(CNC(=O)c2oc(-c3ccc(O)cc3)cc2-c2ccoc2)cc1]

The method for determining purity by HPLC—used throughout these examples—used the following parameters:

| Column | Phenomenex; Luna C18(2); 150 × 4.6 mm; 3 μm | | |
|---|---|---|---|
| Mobile Phase | A: 0.1 HCOOH in H₂O | | |
| | BL 0.1% HCOOH in acrylonitrile (ACN) | | |
| | Time | A | B |
| | (min) | (%) | (%) |
| Gradient | 0.0 | 90 | 10 |
| | 2.0 | 90 | 10 |
| | 15.0 | 10 | 90 |
| | 20.0 | 10 | 90 |
| | 20.1 | 90 | 10 |
| | 23.0 | 90 | 10 |

| | |
|---|---|
| Flow rate | 1.0 ml/min |
| Column temperature | Ambient |
| Detection | UV-vis 254 nm; bandwidth 5 nm; data collection |
| | rate 10 Hz (0.1 s); average: on; ref. |
| | Wavelength: off |
| Injection volume | 10 μl |

It was noted that the dominant impurity was a carboxylic acid component, present at RRT 1.12 at >1.5%. A series of extractions were attempted to remove this and other impurities. The solvent tetrahydrofuran (THF) was used to facilitate dissolution, and to allow selective wash-out of the carboxylic acid. A partition between THF, water and saturated sodium bicarbonate solution resulted in a brown solution that could be phase-separated by the inclusion of brine. A single pass using this methodology improved the purity of QTX125 to >96% as key impurities were selectively removed into the liquors (Table 2).

TABLE 2
Removal of impurities following partitioning

| RRT | 0.89 | 1.12 |
|---|---|---|
| % | 0.30 | 1.20 |

As described now, a rapid evaporation was attempted to provide the amorphous phase from a solution of QTX125. Approximately 5.8 g of QXT125 was dissolved in a mixture of THF and water (1:1, 200 mL). To this was added 20 ml of saturated sodium bicarbonate to yielding a single phase solution. Brine (50 ml) was added to yield a partition, the aqueous phase was separated. The aqueous was then back extracted with ethyl acetate (20 ml) and the combined organics reduced in vacuo. The resulting pale brown solids were slurried in water (15 ml), filtered and dried in vacuo at 45° C. to yield a grey/brown powder (80%, JN572C dry).

A predominantly amorphous material was successfully isolated.

As an alternative, crash precipitation was attempted by dissolving crude QTX125 (1 g) in hot DMSO (3 mL) which was then added to into ice cold water (20 mL). After stirring for 10 minutes the solids were filtered, returned to the vessel and slurried in water (15 ml), filtered, washed (15 ml) and pulled dry. The resulting grey/brown solid was dried at 45° C. in vacuo (98% yield, 95.77%, (rrt 0.89/0.90%, rrt 1.12/1.25%)). This provided a material that could be filtered and was of very low crystallinity (predominantly amorphous). It was also noted that this method of isolation and potential purification, whilst improving the purity to >95%, did increase a key impurity (identified at RRT 1.03, increasing from 0.78% to 0.97%).

The THF partition and crash precipitation methodologies were next combined. Approximately 5.8 g of crude QTX125 was dissolved in a mixture of THF (100 mL) and water (100 mL). 20 mL of saturated sodium bicarbonate was added to this mix, to yield a single-phase solution. Brine (50 mL) was then added to yield a partition, to separate the aqueous phase, and the process was repeated.

The aqueous phase was then back-extracted with ethyl acetate (20 mL) and the combined organics were reduced in vacuo. The resulting pale brown solids were slurried in water (15 mL), filtered and dried in vacuo at 45° C. to yield a grey/brown powder. A hot solution of crude QTX125 in DMSO (1 g, 3 mL) was next polish-filtered into ice cold water (20 mL) to induce a rapid precipitation. After stirring for 10 minutes, the solids were filtered, returned to the vessel and slurried in water (15 mL), filtered, washed (15 mL) and pulled dry. The final purity of QTX125 was noted to be slightly reduced, at >95.3%. However, of greater significance was the identification of a new polymorph of QTX125. This entity, QTX125 ‘Form 2’ was well defined by PXRD, as shown in FIGS. 2A-C. In all examples, PXRD data was recorded using a PANalytical X′Pert PRO diffractometer with a PixCEL detector used in transmission geometry (wavelength of X-rays 1.54056 Å, Cu Kα radiation, at 40 kV and 40 mA) in a range of 2-38° 2θ. A typical step width of 0.013° 2θ and a 25 s measuring time per step was used.

In brief, the profile of Form 2 observed by DSC is broadly similar to that of crude QTX125, with a preceding minor exotherm ahead of the major decomposition event. The degradation of Form 2 is initiated at a higher temperature than crude QTX125, as illustrated by the DSC overlay shown in FIG. 2C. Note that the minor endothermic transitions have also been removed. The peak for Form 2 is observed at approximately 10° C. higher than that of crude QTX125 (FIG. 2C) therefore suggesting that Form 2 may be thermodynamically stable than crude QTX125, and this is supported by the PXRD profile. The combined DSC and TGA traces of Form 2 as shown in FIG. 2D demonstrate the behaviour of the new entity, with the exothermic decomposition events of both traces overlaying well.

### Example 2—QTX125 Crystallisation and Intermediate Process Scale-Up

Given that the purity of QTX125 was improved by ethanol/water slurry, and that a new solid product was identified (Example 1), crystallization of QTX125 was examined. It should be noted here that “% th” refers to % of theoretical yield; “uncorrected” means that no purity correction is made so that purity is assumed to be 100% when calculating yield.

250 mg QTX125 was suspended in 12 volumes of ethanol/water 5% (v/v), at reflux, and 8 volumes of THF were charged in aliquots to give a solution containing lumps of QTX125. This was clarified into a crystallisation tube and allowed to stand sealed for 48 hours, with no observed solid formation. The solution was agitated and heated to 50° C. under a gentle stream of nitrogen to concentrate the solution. Once solids were observed in suspension, the mixture was cooled to ambient temperature and isolated by filtration and dried in vacuo at 45° C. overnight. A total of 185 mg QTX125 was recovered (74% th., uncorrected). Chemical purity was assessed by HPLC as 98.43%, containing 0.44% acid impurity (RRT 1.12). A ¹H NMR assay in DMSO was used to assess residual solvent levels. Purity was identified as 98%, containing residual ethanol at 0.62% and THF at 0.43%.

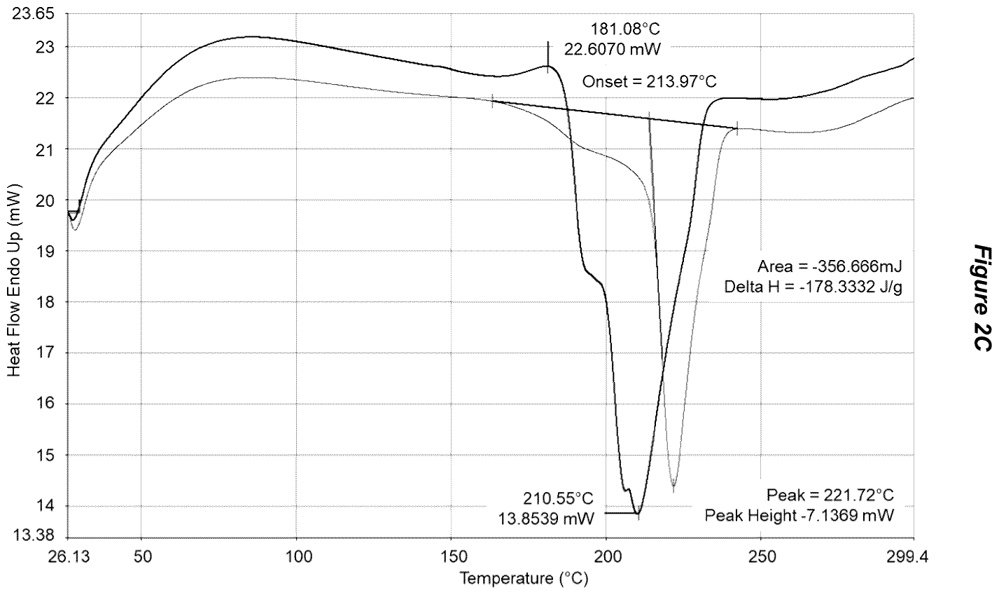

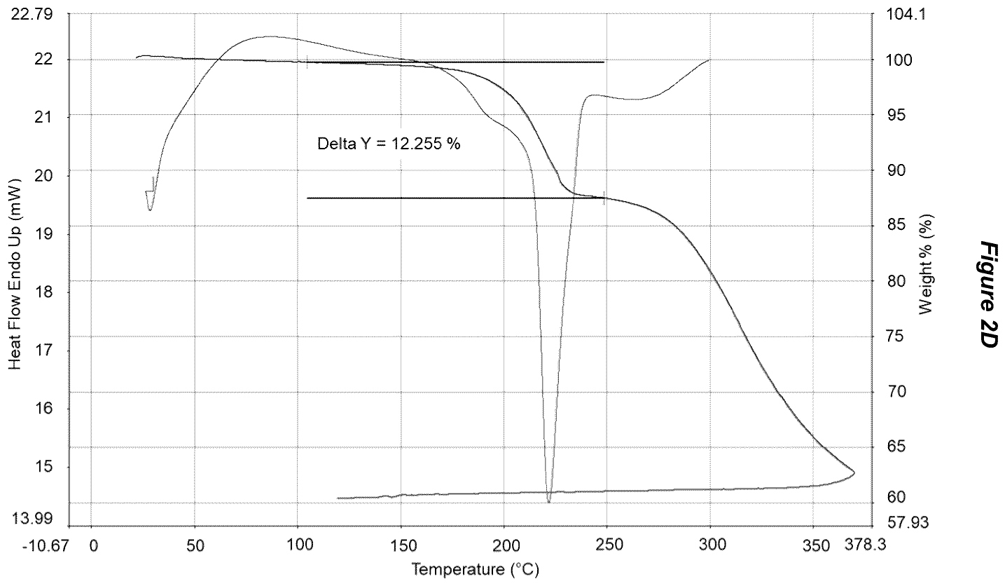

As shown in FIGS. 3A and 3B, the crystalline species of QTX125 was identified as Form 2. Thermal analysis shows the typical exothermic decomposition of the crystalline form, in this instance a small endothermic event at 233° C. is notable. This may be indicative of an initial melt transformation. TGA analysis concurs with the decomposition statement (as reported above for Form 2). The sample is relatively solvent and water free.

In summary, small-scale crystallisation of QTX125 has been successfully demonstrated using ethanol, THF and water mixture. Improved chemical purity is demonstrated by HPLC and via the ¹H NMR assay. Although unoptimized, the small-scale crystallisation process was considered the most suitable method to purify QTX125 Form 2.

### Scale-Up (Reaction I)

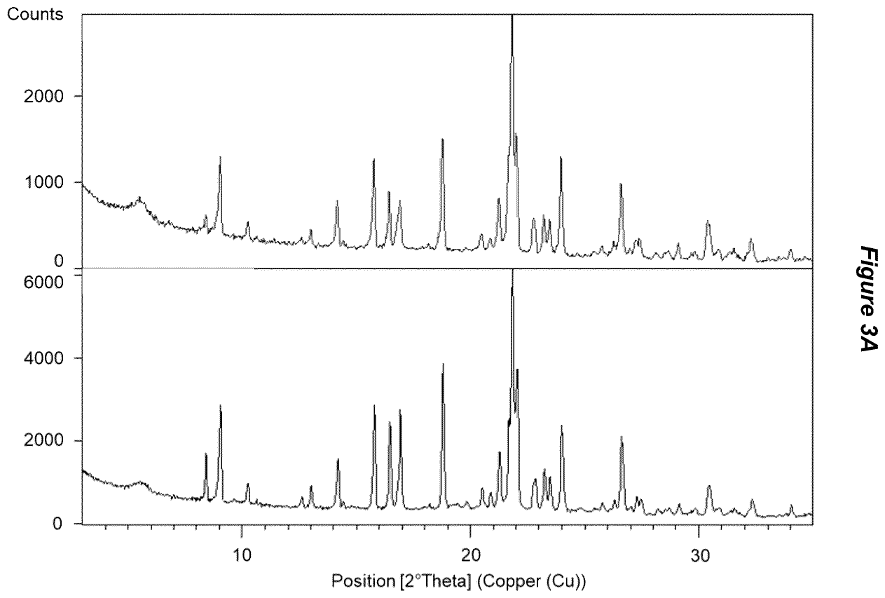

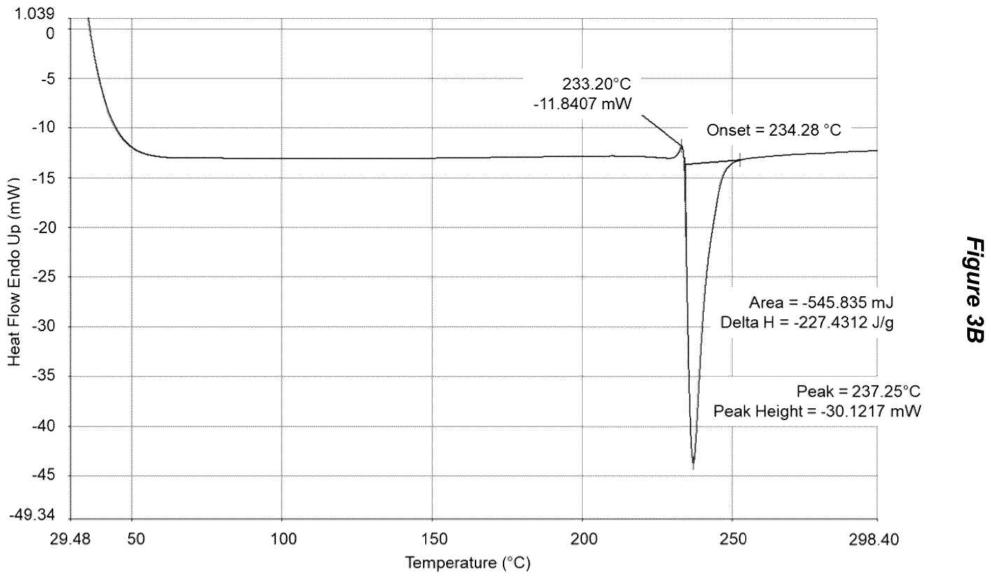

The small-scale crystallisation method was scaled-up to produce material for the stability and solubility investigations (Examples 3, 4 and 5). A total of 2.5668 g crude QTX125 was used, and 10 volumes of THF were required to give a hazy brown mixture prior to clarification. The isolated solid was dried in vacuo at 50° C., and a total of 1.0617 g QTX125 was recovered (41.36% th., uncorrected). Chemical purity was assessed by HPLC as 98.21%, containing 0.19% acid impurity (RTT 1.12). A ¹H NMR assay in DMSO was used to assess residual solvent levels. Purity was identified as 98%, containing residual ethanol at 0.26% and THF at 0.17%. As shown in FIGS. 4A to 4C, the crystalline species of QTX125 was again identified as Form 2. The DSC thermographs are almost identical to that of Form 2 isolated from the small-scale crystallisation, with no low temperature events and characterised by a minor exotherm, endotherm and the main exotherm at 235° C. In addition, the TGA thermograph reveals no weight reduction up to 180° C., followed by an 8.5% weight reduction coincident with the main exotherm. The product of the crystallisation is essentially solvent free, as corroborated by NMR.

In summary, the crystallisation of QTX125 had been successfully repeated to produce Form 2 with high chemical purity, excellent thermal characteristics and very little residual solvent (ethanol content was within the ICH limit and THF was not above 720 ppm). This is the “first scale up” referred to elsewhere herein.

### Scale-Up (Reaction II)

Considering the low yield of Form 2 from the first scale-up reaction, the crystallisation protocol was repeated with a modified solvent regime in order to assess whether the recovery of QTX125 could be improved, whilst maintaining high chemical purity. This is the “second scale up” referred to elsewhere herein.

A total of 2.571 g crude QTX125 was suspended in 12.4 volumes of ethanol and 6 volumes of THF, with agitation and heated to reflux. 4 mL deionised water was charged at reflux to give a solution which was clarified into a crystallisation flask at 80° C. The solution was agitated and cooled to 50° C. where upon some solid was observed to form. A nitrogen stream was applied to the solution to concentrate the mixture until a solid in suspension was observed. The mixture was returned to 50° C. and then cooled to ambient temperature gradually. The solid was isolated by filtration and dried in vacuo at 50° C. and a total of 1.7557 g QTX125 was recovered (68.29% th., uncorrected). Chemical purity was assessed by HPLC as 97.86%, containing 0.36% acid impurity (RTT 1.12). A ¹H NMR assay in DMSO was used to assess residual solvent levels. Purity was identified as 97%, containing residual ethanol at 0.48% and THF at 0.34%. The crystalline species of QTX125 isolated via this method was confirmed as Form 2 via PXRD (FIG. 5A). As shown in FIG. 5B, the DSC and TGA thermographs are almost identical to that of QTX125 isolated from the first scale-up reaction, with no low temperature events and characterised by a minor exotherm, endotherm and the main exotherm at 238° C. with coincident weight reduction of 8.3%.

An alternative scaled-up procedure is as follows:

QXT125, 1 wt (g per mL of solvent) was suspended in water (4 vol i.e. 4 mL per 1 g of QTX125) at 100° C. Propanol, 5 vol (i.e. 5 mL per 1 g of QTX125), ethanol, 1 vol (i.e. i.e. 1 mL per 1 g of QTX125), THF, 3 vol (i.e. 3 mL per 1 g of QTX125), and dioxane, 0.867 vol (i.e. 0.867 mL per 1 g of QTX125) were added. The solution was clarified into a crystallisation vessel at 100° C. and allowed to cool with agitation, during which solid was observed to form. The mixture was agitated overnight.

The solid was isolated by filtration, and the filter cake treated with the following solvents by displacement:
Ethanol, 2 vol (i.e. 2 mL per 1 g of QTX125)
Water, 2 vol (i.e. 2 mL per 1 g of QTX125)
Ethanol, 2 vol (i.e. 2 mL per 1 g of QTX125)

The solid was dried in vacuo at 50° C. overnight. Recovery: 0.5916 g, 55% th. uncorr. 1H NMR, DMSO, concordant with structure. Residual solvents: dioxane, 0.21%, propanol/ethanol, 0.14%.

CP by HPLC. Recovery: 9.26 g, 62.06% th. uncorr. 1H NMR, DMSO, concordant with structure. Residual solvents: present but not quantifiable. CP by HPLC, 99.4 area %. No single impurity greater than 0.5 area %. PXRD pattern, concordant with Form 2. The DSC thermograph, concordant with Form 2 with a single exotherm at 242° C.

### Example 3—Crystalline Form of an Adduct of a Compound of Formula I

L-Lysine, 2 equiv, 2M, was clarified into agitated ethanol, 43 ml, 48 vol (i.e. 48 mL per 1 g of QTX125), at 60° C. which had also been clarified. QXT125, 1 wt (g per mL), 1 equiv, 0.9009 g, was dissolved in THF, 3.6 ml, 4 vol (i.e. 4 mL per 1 g of QTX125), and water, 0.55 ml, 0.6 vol (i.e. 0.6 mL per 1 g of QTX125), and clarified into the L-lysine solution at 60° C. and cooled to 50° C. for 0.5 hours. The mixture was allowed to cool with agitation over 18 hours and agitation continued at ambient temperature for 24 hours. The solid was recovered by filtration and the filter cake washed with ethanol, 2×10 ml, and then dried in vacuo at 50° C.

Recovery: 1.4743 g, 96.24% th. uncorr. Chemical purity by HPLC: 96.85 area % (0.35% acid impurity, RRT 1.12). ¹H NMR assay in DMSO/D₂O: 96%, containing residual ethanol, 3.02%, and the stoichiometry of QTX125 to L-Lysine is 1:2.

PXRD (FIG. 9), TGA and DSC (FIG. 10) indicate high crystallinity.

### Example 4—Assessing the Photostability of QTX125

The photostability of novel crystalline forms of QTX125 was assessed at solid state and in solution. Where ‘forced illumination’ conditions were used, samples were illuminated at 12 Klux/hour and 2.8 UV W/m²/hour. Samples were stored at a temperature of 30° C. Following incubation, the chemical purity of QTX125 was measured by HPLC.

The following index is provided to aid navigation of the data presented in Tables 3.1.1 to 3.3.6:
3.1.x—Assessment of the photostability of QTX125 Form 2.
3.2.x—Assessment of the photostability of amorphous QTX125.
3.3.x—Assessment of the photostability of the QTX125 1:2 L-Lysine adduct.
3.x.1—Samples (at solid state) stored in unsealed clear glass bottles, subject to forced illumination.
3.x.2—Samples (at solid state) sealed under nitrogen in clear glass bottles, subject to forced illumination.
3.x.3—Samples (at solid state) sealed under nitrogen in amber glass bottles, illuminated under ambient laboratory conditions
3.x.4—Samples (at solid state) sealed under nitrogen in amber glass bottles, subject to forced illumination.
3.x.5—Samples (in solution) sealed under nitrogen in amber glass bottles, illuminated under ambient laboratory conditions.
3.x.6—Samples (in solution) sealed under nitrogen in amber glass bottles, subject to forced illumination.

Summary data comparing the photostability of the novel crystalline forms of QTX125 following 171 hours of incubation is provided in Tables 3.4.1 to 3.4.4.

### Assessment of the Photostability of QTX125 Form 2

TABLE 3.1.1.
Photostability of QTX125 Form 2 was assessed at solid-state, stored
in unsealed clear glass bottles. Samples were subject to forced
illumination. Values provided are percentage (%) HPLC peak area.

| | | RRT | | | | | |
|---|---|---|---|---|---|---|---|
| | | QTX125 | | | | | |
| | | 1.00 | 1.04 | 1.12 | — | — | — |
| Time | 0 | 98.21 | 0.98 | — | — | — | — |
| (Hours) | 3 | 97.68 | 1.38 | — | — | — | — |
| | 26 | 95.59 | 2.38 | — | — | — | — |
| | 74 | 90.53 | 4.90 | — | — | — | — |
| | 171 | 83.78 | 9.15 | 0.52 | — | — | — |

Form 2 is shown to degrade to two principal components over the course of 171 hours. The solid material was observed to change in colour from off white to dark green.

TABLE 3.1.2.
Photostability of QTX125 Form 2 was assessed at solid-
state, sealed under nitrogen in clear glass bottles.
Samples were subject to forced illumination. Values
provided are percentage (%) HPLC peak area.

| | | RRT | | | | | |
|---|---|---|---|---|---|---|---|
| | | QTX125 | | | | | |
| | | 1.00 | 1.04 | 1.08 | 1.11 | 1.20 | — |
| Time | 0 | 98.21 | 0.98 | — | — | — | — |
| (Hours) | 3 | 97.78 | 1.29 | — | — | — | — |
| | 26 | 96.16 | 2.16 | — | — | — | — |
| | 74 | 84.98 | 5.99 | 1.24 | 0.52 | 0.66 | — |
| | 171 | 82.33 | 9.95 | 0.41 | 0.86 | 0.22 | — |

Form 2 is shown to degrade to two principal components over the course of 171 hours (at RRTs 1.04 and 1.11). The data also indicates that secondary degradation is taking place, as new impurities are identified, at RRTs 1 0.08 and 1 0.20 entities. The solid material was observed to change in colour from off white to dark green.

TABLE 3.1.3.
Photostability of QTX125 Form 2 was assessed at solid-
state, sealed under nitrogen in amber glass bottles. Samples
were illuminated under ambient laboratory conditions.
Values provided are percentage (%) HPLC peak area.

| | | RRT | | | | | |
|---|---|---|---|---|---|---|---|
| | | QTX125 | | | | | |
| | | 1.00 | 1.04 | — | — | — | — |
| Time | 0 | 98.21 | 0.98 | — | — | — | — |
| (Hours) | 3 | 97.35 | 1.06 | — | — | — | — |
| | 26 | 98.11 | 1.07 | — | — | — | — |
| | 74 | 98.83 | 0.55 | — | — | — | — |
| | 171 | 98.12 | 1.21 | — | — | — | — |

Form 2 shows little evidence of degradation when stored in these conditions.

TABLE 3.1.4.
Photostability of QTX125 Form 2 was assessed at solid-
state, sealed under nitrogen in amber glass bottles.
Samples were subject to forced illumination. Values
provided are percentage (%) HPLC peak area.

| | | RRT | | | | | |
|---|---|---|---|---|---|---|---|
| | | QTX125 | | | | | |
| | | 1.00 | 1.04 | 1.12 | — | — | — |
| Time | 0 | 98.21 | 0.98 | — | — | — | — |
| (Hours) | 3 | 98.23 | 0.99 | — | — | — | — |
| | 26 | 97.82 | 1.22 | — | — | — | — |
| | 74 | 97.70 | 1.06 | — | — | — | — |
| | 171 | 95.40 | 2.55 | 0.28 | — | — | — |

Form 2 is shown to degrade slightly. The principal degradation components identified are the same as those derived from amorphous QTX125 stored in unsealed clear glass bottles (shown in Table 3.2.1) or sealed under nitrogen (shown in Table 3.2.2). These results indicate that the degradation of Form 2 may be minimised by limiting exposure to light, for example by storing samples in opaque or amber glass containers.

TABLE 3.1.5.
Photostability of QTX125 Form 2 was assessed in solution (THF
and water), sealed under nitrogen in amber glass bottles.
Samples were illuminated under ambient laboratory conditions.
Values provided are percentage (%) HPLC peak area.

| | | RRT | | | | | |
|---|---|---|---|---|---|---|---|
| | | QTX125 | | | | | |
| | | 1.00 | 1.04 | 1.12 | — | — | — |
| Time | 0 | 98.21 | 0.98 | — | — | — | — |
| (Hours) | 3 | 97.50 | 1.06 | — | — | — | — |
| | 26 | 97.65 | 0.93 | — | — | — | — |
| | 171 | 91.10 | 4.81 | 0.83 | — | — | — |

Form 2 is shown to degrade when stored in solution under ambient laboratory conditions. The principal degradation components are the same as those derived from amorphous QTX125 stored in unsealed clear glass bottles (shown in Table 3.2.1) or sealed under nitrogen (shown in Table 3.2.2).

TABLE 3.1.6.
Photostability of QTX125 Form 2 was assessed in solution
(THF and water), sealed under nitrogen in amber glass
bottles. Samples were subject to forced illumination.
Values provided are percentage (%) HPLC peak area.

| | | RRT | | | | | |
|---|---|---|---|---|---|---|---|
| | | QTX125 | | | | | |
| | | 1.00 | 1.04 | 1.12 | — | — | — |
| Time | 0 | 98.21 | 0.98 | — | — | — | — |
| (Hours) | 3 | 97.30 | 1.07 | — | — | — | — |
| | 26 | 96.27 | 1.09 | — | — | — | — |
| | 171 | 81.65 | 3.60 | 0.86 | — | — | — |

Form 2 is shown to degrade when stored in solution under ambient laboratory conditions.

The principal degradation components are the same as those derived from amorphous QTX125 stored in unsealed clear glass bottles (shown in Table 3.2.1) or sealed under nitrogen (shown in Table 3.2.2). The data also indicates that secondary degradation of the component identified at RRT 1.04 may occur.

### Assessment of the Photostability of Amorphous QTX125

TABLE 3.2.1
Photostability of amorphous QTX125 was assessed at solid-state, stored
in unsealed clear glass bottles. Samples were subject to forced
illumination. Values provided are percentage (%) HPLC peak area.

| | | RRT | | | | | |
|---|---|---|---|---|---|---|---|
| | | QTX125 | | | | | |
| | | 1.00 | 1.04 | 1.12 | — | — | — |
| Time | 0 | 96.74 | 1.32 | 0.35 | — | — | — |
| (Hours) | 3 | 95.81 | 1.61 | 0.39 | — | — | — |
| | 50 | 88.70 | 4.64 | 0.63 | — | — | — |
| | 171 | 83.01 | 7.15 | 0.90 | — | — | — |

Amorphous QTX125 degrades under forced illumination to two principal components. The solid material was observed to change in colour from off white/beige to brown.

TABLE 3.2.2
Photostability of amorphous QTX125 was assessed at
solid-state, sealed under nitrogen in clear glass
bottles. Samples were subject to forced illumination.
Values provided are percentage (%) HPLC peak area.

| | | RRT | | | | | |
|---|---|---|---|---|---|---|---|
| | | QTX125 | | | | | |
| | | 1.00 | 1.04 | 1.12 | — | — | — |
| Time | 0 | 96.74 | 1.32 | 0.35 | — | — | — |
| (Hours) | 3 | 95.97 | 1.51 | 0.38 | — | — | — |
| | 50 | 89.64 | 4.38 | 0.60 | — | — | — |
| | 171 | 84.46 | 6.61 | 0.90 | — | — | — |

Amorphous QTX125 degrades under forced illumination to two principal components. The solid material was observed to change in colour from off white/beige to brown.

TABLE 3.23
Photostability of amorphous QTX125 was assessed at solid-
state, sealed under nitrogen in amber glass bottles. Samples
were illuminated under ambient laboratory conditions. Values
provided are percentage (%) HPLC peak area.

| | | RRT | | | | | |
|---|---|---|---|---|---|---|---|
| | | QTX125 | | | | | |
| | | 1.00 | 1.04 | 1.12 | — | — | — |
| Time | 0 | 96.74 | 1.32 | 0.35 | — | — | — |
| (Hours) | 3 | 96.75 | 1.26 | 0.36 | — | — | — |
| | 50 | 96.61 | 0.71 | 0.39 | — | — | — |
| | 171 | 96.16 | 1.46 | 0.39 | — | — | — |

Amorphous QTX125 shows evidence of slight degradation when illuminated under ambient laboratory conditions.

TABLE 3.2.4
Photostability of amorphous QTX125 was assessed at
solid-state, sealed under nitrogen in amber glass
bottles. Samples were subject to forced illumination.
Values provided are percentage (%) HPLC peak area.

| | | RRT | | | | | |
|---|---|---|---|---|---|---|---|
| | | QTX125 | | | | | |
| | | 1.00 | 1.04 | 1.12 | — | — | — |
| Time | 0 | 96.74 | 1.32 | 0.35 | — | — | — |
| (Hours) | 3 | 96.94 | 1.04 | 0.35 | — | — | — |
| | 50 | 96.12 | 1.09 | 0.38 | — | — | — |
| | 171 | 94.43 | 1.70 | 0.47 | — | — | — |

Amorphous QTX125 degrades slightly under forced illumination conditions. The principal components degradation components are the same as those derived from amorphous QTX125 stored in unsealed clear glass bottles (shown in Table 3.2.1) or sealed under nitrogen (shown in Table 3.2.2). These results indicate that the degradation of amorphous QTX125 may be minimised by limiting exposure to light, for example by storing samples in opaque or amber glass containers.

TABLE 3.2.5
Photostability of amorphous QTX125 was assessed in solution
(THF and water), sealed under nitrogen in amber glass bottles.
Samples were illuminated under ambient laboratory conditions.
Values provided are percentage (%) HPLC peak area.

| | | RRT | | | | | |
|---|---|---|---|---|---|---|---|
| | | QTX125 | | | | | |
| | | 1.00 | 1.04 | 1.12 | — | — | — |
| Time | 0 | 96.74 | 1.32 | 0.35 | — | — | — |
| (Hours) | 3 | 92.10 | 0.94 | 0.48 | — | — | — |
| | 50 | 95.89 | 1.03 | 0.54 | — | — | — |
| | 171 | 91.98 | 2.30 | 1.14 | — | — | — |

Amorphous QTX125 degrades when in solution under ambient laboratory conditions. The principal degradation components are the same as those identified in Tables 3.2.1 and 3.2.2.

TABLE 3.2.6
Photostability of amorphous QTX125 was assessed in solution
(THF and water), sealed under nitrogen in amber glass
bottles. Samples were subject to forced illumination.
Values provided are percentage (%) HPLC peak area.

| | | RRT | | | | | |
|---|---|---|---|---|---|---|---|
| | | QTX125 | | | | | |
| | | 1.00 | 1.04 | 1.12 | — | — | — |
| Time | 0 | 96.74 | 1.32 | 0.35 | — | — | — |
| (Hours) | 3 | 96.31 | 0.92 | 0.46 | — | — | — |
| | 50 | 94.55 | 1.27 | 0.55 | — | — | — |
| | 171 | 87.29 | 2.33 | 1.03 | — | — | — |

Amorphous QTX125 degrades when in solution under forced illumination conditions. The principal degradation components are the same as those identified in Tables 3.2.1 and 3.2.2.

### Assessment of the Photostability of the QTX125 1:2 L-Lysine Adduct

TABLE 3.3.1
Photostability of the QTX125 1:2 L-Lysine adduct was
assessed at solid-state, stored in unsealed clear glass
bottles. Samples were subject to forced illumination.
Values provided are percentage (%) HPLC peak area.

| | | RRT | | | | | |
|---|---|---|---|---|---|---|---|
| | | QTX125 | | | | | |
| | | 1.00 | 1.04 | 1.12 | — | — | — |
| Time | 0 | 96.83 | 1.15 | — | — | — | — |
| (Hours) | 3 | 95.51 | 1.98 | — | — | — | — |
| | 50 | 88.96 | 6.16 | 3.08 | — | — | — |
| | 171 | 84.14 | 9.20 | 4.38 | — | — | — |

The QTX125 1:2 L-Lysine adduct degrades under forced illumination to two principal components. A change in the colour of the solid material was not identified.

TABLE 3.3.2
Photostability of the QTX125 1:2 L-Lysine adduct was assessed
at solid-state, sealed under nitrogen in clear glass
bottles. Samples were subject to forced illumination.
Values provided are percentage (%) HPLC peak area.

| | | RRT | | | | | |
|---|---|---|---|---|---|---|---|
| | | QTX125 | | | | | |
| | | 1.00 | 1.04 | 1.12 | — | — | — |
| Time | 0 | 96.83 | 1.15 | — | — | — | — |
| (Hours) | 3 | 95.57 | 1.90 | 0.65 | — | — | — |
| | 50 | 89.67 | 5.79 | 2.74 | — | — | — |
| | 171 | 85.21 | 9.04 | 3.94 | — | — | — |

The QTX125 1:2 L-Lysine adduct degrades under forced illumination to two principal components. A change in the colour of the solid material was not identified.

TABLE 3.3.3
Photostability of the QTX125 1:2 L-Lysine adduct was assessed
at solid-state, sealed under nitrogen in amber glass bottles.
Samples were illuminated under ambient laboratory conditions.
Values provided are percentage (%) HPLC peak area.

| | | RRT | | | | | |
|---|---|---|---|---|---|---|---|
| | | QTX125 | | | | | |
| | | 1.00 | 1.04 | 1.12 | — | — | — |
| Time | 0 | 96.83 | 1.15 | — | — | — | — |
| (Hours) | 3 | 96.82 | 1.21 | — | — | — | — |
| | 50 | 97.84 | 0.58 | — | — | — | — |
| | 171 | 97.29 | 1.30 | 0.35 | — | — | — |

The QTX125 1:2 L-Lysine adduct shows little evidence of degradation when illuminated under ambient laboratory conditions.

TABLE 3.3.4
Photostability of the QTX125 1:2 L-Lysine adduct was
assessed at solid-state, sealed under nitrogen in amber
glass bottles. Samples were subject to forced illumination.
Values provided are percentage (%) recovery.

| | | RRT | | | | | |
|---|---|---|---|---|---|---|---|
| | | QTX125 | | | | | |
| | | 1.00 | 1.04 | 1.12 | — | — | — |
| Time | 0 | 96.83 | 1.15 | | — | — | — |
| (Hours) | 3 | 96.92 | 1.18 | | — | — | — |
| | 50 | 97.07 | 1.01 | 0.41 | — | — | — |
| | 171 | 96.27 | 1.74 | 0.59 | — | — | — |

The QTX125 1:2 L-Lysine adduct degrades slightly under forced illumination conditions. The principal degradation components identified are the same as those derived from the L-Lysine adduct stored in unsealed clear glass bottles (Table 3.3.1) or sealed under nitrogen (shown in Table 3.3.2). These results indicate that degradation of the adduct may be minimised by limiting exposure to light, for example by storing samples in opaque or amber glass containers.

TABLE 3.3.5
Photostability of the QTX125 1:2 L-Lysine adduct was assessed
in solution (THF and water), sealed under nitrogen in amber
glass bottles. Samples were illuminated under ambient laboratory
conditions. Values provided are percentage (%) recovery.

| | | RRT | | | | | |
|---|---|---|---|---|---|---|---|
| | | QTX125 | | | | | |
| | | 1.00 | 1.04 | 1.12 | — | — | — |
| Time | 0 | 96.83 | 1.15 | — | — | — | — |
| (Hours) | 3 | 97.08 | 0.80 | 0.37 | — | — | — |
| | 50 | 94.35 | 0.54 | 1.03 | — | — | — |
| | 171 | 93.18 | 1.50 | 0.97 | — | — | — |

The QTX125 1:2 L-Lysine adduct degrades when in solution and illuminated under ambient laboratory conditions. The principal degradation components are the same as those identified in Tables 3.3.1 and 3.3.2.

TABLE 3.3.6
Photostability of the QTX125 1:2 L-Lysine adduct was assessed
in solution (THF and water), sealed under nitrogen in amber
glass bottles. Samples were subject to forced illumination.
Values provided are percentage (%) HPLC peak area.

| | | RRT | | | | | |
|---|---|---|---|---|---|---|---|
| | | QTX125 | | | | | |
| | | 1.00 | 1.04 | 1.12 | — | — | — |
| Time | 0 | 96.83 | 1.15 | — | — | — | — |
| (Hours) | 3 | 98.01 | 0.71 | 0.33 | — | — | — |
| | 50 | 84.17 | 1.03 | 2.14 | — | — | — |
| | 171 | 89.96 | 2.82 | 1.17 | — | — | — |

The QTX125 1:2 L-Lysine adduct degrades when in solution and subject to forced illumination. The principal degradation components are the same as those identified in Tables 3.3.1 and 3.3.2. These data indicate that degradation is enhanced if the L-Lysine adduct is stored in solution, which is therefore not advised.

### Summary Data

Summary data comparing the photostability of QTX125 is provided below, in Tables 3.4.1 to 3.4.4.

TABLE 3.4.1
Summary data showing the photostability of QTX125 following
171 hours of incubation. All samples were stored at solid-
state, sealed under nitrogen in amber glass bottles and
illuminated under ambient laboratory conditions. Values
provided are percentage (%) HPLC peak area.

| | | RRT | | | | |
|---|---|---|---|---|---|---|
| | | QTX125 | | | | |
| | | 1.00 | 1.04 | 1.12 | — | — |
| QTX125 | Form 2 | 98.12 | 1.21 | | — | — |
| Entity | Amorphous | 96.16 | 1.46 | 0.39 | — | — |
| | 1:2 L-Lysine adduct | 97.29 | 1.30 | 0.35 | — | — |

In summary, all QTX125 entities show little evidence of degradation when stored in amber glass bottles and illuminated under ambient laboratory conditions.

TABLE 3.4.2
Summary data showing the photostability of QTX125 following 171
hours of incubation. All samples were stored at solid-state, sealed
under nitrogen in amber glass bottles, and subject to forced illumination.
Values provided are percentage (%) HPLC peak area.

| | | RRT | | | | |
|---|---|---|---|---|---|---|
| | | QTX125 | | | | |
| | | 1.00 | 1.04 | 1.12 | — | — |
| QTX125 | Form 2 | 95.40 | 2.55 | 0.28 | — | — |
| Entity | Amorphous | 94.43 | 1.70 | 0.47 | — | — |
| | 1:2 L-Lysine adduct | 96.27 | 1.74 | 0.59 | — | — |

In summary, all QTX125 entities show little evidence of degradation when stored in amber glass bottles and illuminated under ambient laboratory conditions, although novel crystalline forms of QTX125 (i.e., Form 2 and the 1:2 L-Lysine adduct) appear to display enhanced photostability as compared to amorphous QTX125.

TABLE 3.4.3
Summary data showing the photostability of QTX125 following
171 hours of incubation. All samples were stored in
solution, sealed under nitrogen in amber glass bottles,
and illuminated under ambient laboratory conditions.
Values provided are percentage (%) HPLC peak area.

| | | RRT | | | | |
|---|---|---|---|---|---|---|
| | | QTX125 | | | | |
| | | 1.00 | 1.04 | 1.12 | — | — |
| QTX125 | Form 2 | 91.10 | 4.81 | 0.83 | — | — |
| Entity | Amorphous | 91.98 | 2.30 | 1.14 | — | — |
| | 1:2 L-Lysine adduct | 93.18 | 1.50 | 0.97 | — | — |

In summary, the L-Lysine 1:2 adduct displays enhanced photostability in solution as compared to amorphous QTX125 and Form 2.

TABLE 3.4.4
Summary data showing the photostability of QTX125 following 171
hours of incubation. All samples were stored in solution, sealed
under nitrogen in amber glass bottles, and subject to forced illumination.
Values provided are percentage (%) HPLC peak area.

| | | RRT | | | | |
|---|---|---|---|---|---|---|
| | | QTX125 | | | | |
| | | 1.00 | 1.04 | 1.12 | — | — |
| QTX125 | Form 2 | 81.65 | 3.60 | 0.86 | — | — |
| Entity | Amorphous | 87.29 | 2.33 | 1.03 | — | — |
| | 1:2 L-Lysine adduct | 89.96 | 2.82 | 1.17 | — | — |

In summary, the L-Lysine 1:2 adduct displays enhanced photostability in solution as compared to amorphous QTX125, or Form 2.

### Conclusions

These data demonstrate that at solid state, Form 2 and the 1:2 L-Lysine adduct of QTX125 display improved photostability as compared to amorphous QTX125. In addition, the 1:2 L-Lysine adduct displays improved photostability when stored in solution, as compared to both QTX125 Form 2 and amorphous QTX125.

### Example 5—Assessing the Aqueous Solubility of QTX125

The solubility of novel crystalline forms of QTX125 was assessed in the following aqueous solutions: (i) phosphate buffer, pH 3.5, 0.9% w/v sodium chloride; (ii) phosphate buffer, pH 6.5, 0.9% w/v sodium chloride; (iii) acetate buffer, pH 4.5, 0.9% w/v sodium chloride; (iv) citro-phosphate buffer, pH 4.5, 0.9% w/v sodium chloride; (v) sodium chloride, 0.9% w/v and (vi) deionised water. All assays were completed at 37° C.

In brief, 30 mg QTX125 was dispensed into vessels followed by 5 mL of the appropriate aqueous buffer. Mixtures were suspended by agitation and heated to 37° C. At various fixed intervals, an 0.4 mL aliquot of the suspension was removed, filtered and diluted for examination by HPLC. A single HPLC reference sample was used.

### Assessment of the Aqueous Solubility of QTX125 Form 2

TABLE 4.1
The solubility of QTX125 Form 2 was assessed in
aqueous buffers (i) to (vi). Values provided are
the concentration of QTX125, measured in mg/mL.

| | | Buffer | | | | | |
|---|---|---|---|---|---|---|---|
| | | (i) | (ii) | (iii) | (iv) | (v) | (vi) |
| Time | 0 | 0 | 0 | 0 | 0 | 0 | 0 |
| (Hours) | 1 | 0.00077 | 0.00033 | 0.00025 | 0.00032 | 0.00068 | 0.0178 |
| | 4 | 0.00015 | 0.00015 | 0.00029 | 0.00017 | 0.00026 | 0.02239 |
| | 7 | 0.0005 | 0.00034 | 0.00019 | 0.00017 | 0.00022 | 0.02782 |
| | 24 | 0.00176 | 0.00063 | 0.0005 | 0.0004 | 0.00081 | 0.01656 |

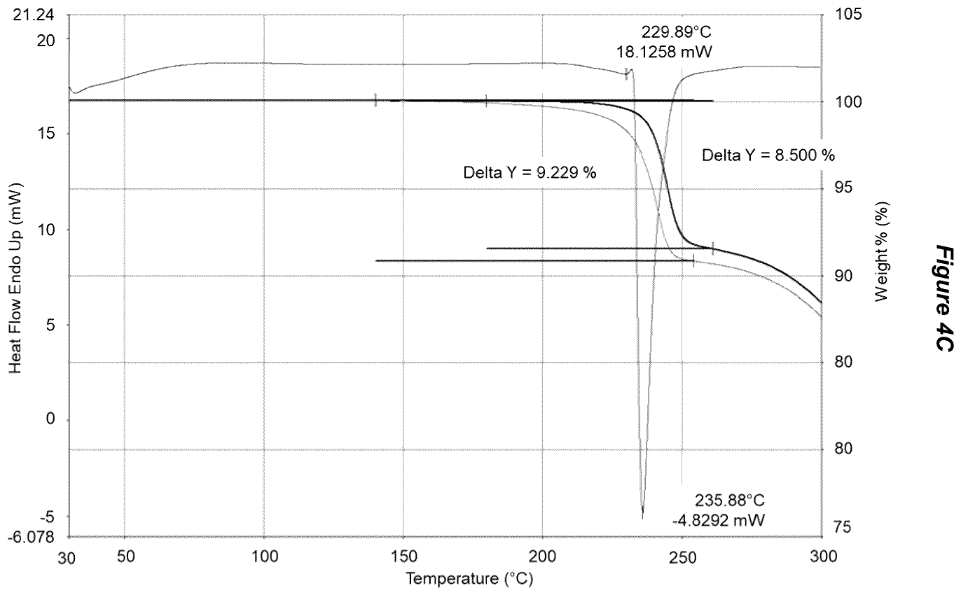

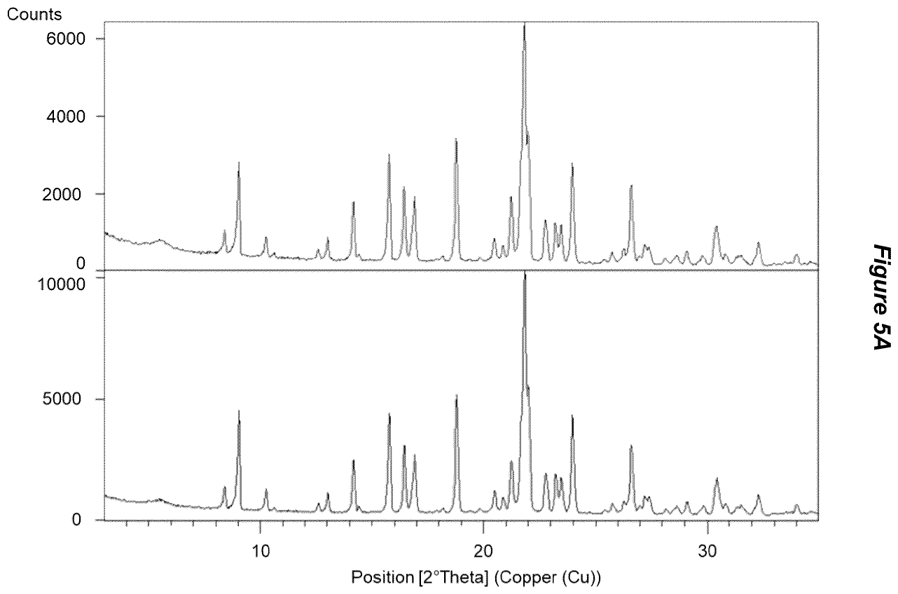

The data presented in Table 4.1 are visualised in FIGS. 6A and 6B. In summary, the solubility of Form 2 in phosphate buffers pH 3.5 (i) and pH 6.5 (ii), in acetate buffer pH 4.5 (iii), in citro-phosphate buffer pH 4.5 (iv), and in sodium chloride 0.9% w/v (v) is very low. Concentration values range from 0.15×10⁻³ to 1.76×10⁻³ mg·ml⁻¹. The solubility profiles follow the same pattern, showing an initial spike in solubility which drops and then increases.

In contrast, the solubility of Form 2 in deionised water (vi) rapidly increases and passes through a maximum at 7 hours, followed by a gradual fall. PXRD analysis of the solid recovered at 24 hours confirmed the entity as Form 2.

### Assessment of the Aqueous Solubility of the QTX125 1:2 L-Lysine Adduct

TABLE 4.2
The solubility of the QTX125 1:2 L-Lysine adduct was assessed
in aqueous buffers (i) to (vi). Values provided are the
concentration of QTX125, measured in mg/mL.

| | | Buffer | | | | | |
|---|---|---|---|---|---|---|---|
| | | (i) | (ii) | (iii) | (iv) | (v) | (vi) |
| Time | 0 | 0 | 0 | 0 | 0 | 0 | 0 |
| (Hours) | 1 | 0.00028 | 0.00032 | 0.00025 | 0.00033 | 0.02981 | 0.0245 |
| | 4 | 0.00033 | 0.00024 | 0.00035 | 0.00029 | 0.01691 | 0.01622 |
| | 7 | 0.00023 | 0.00039 | 0.00035 | 0.00037 | 0.00851 | 0.01014 |
| | 24 | 0.00034 | 0.00028 | 0.00033 | 0.00027 | 0.00452 | 0.00776 |

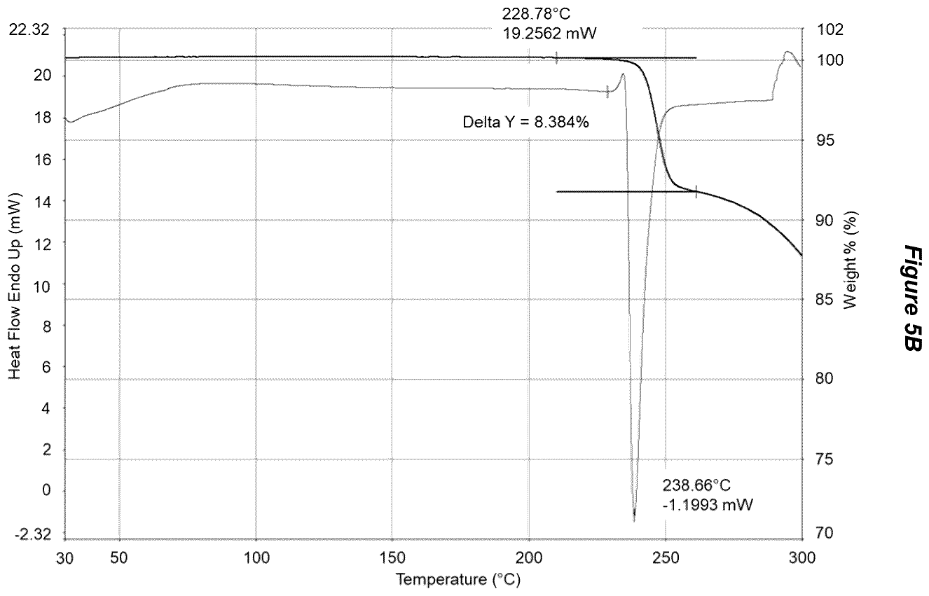

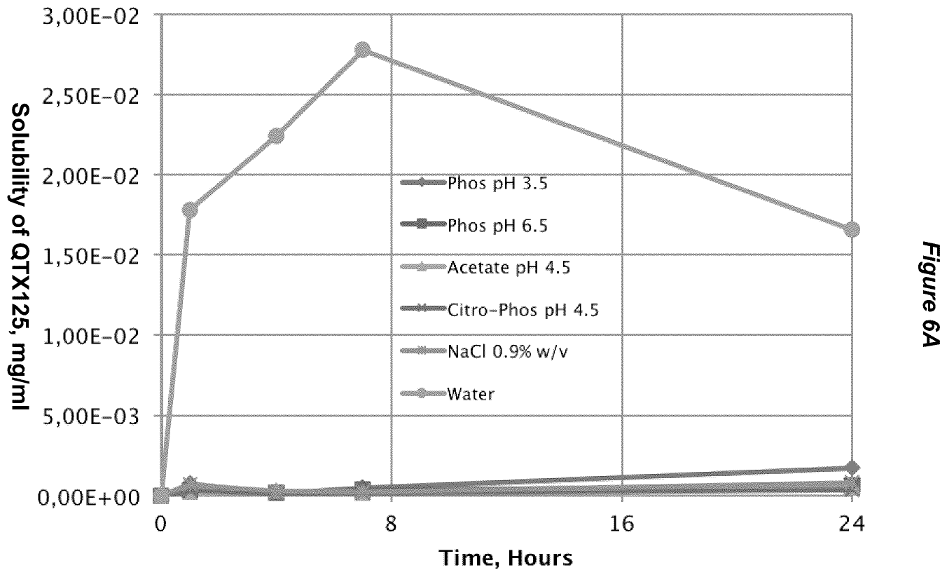

The data presented in Table 4.3 are visualised in FIGS. 7A and 7B. In summary, solubility of the adduct in phosphate buffers pH 3.5 (i) and pH 6.5 (ii), in acetate buffer pH 4.5 (iii), and in citro-phosphate buffer pH 4.5 (iv) is very low. Concentration values range from 0.23×10⁻³ to 0.39×10⁻³ mg·ml⁻¹. Solubility is shown to increase and decrease over time, as a consequence of the adduct dissolving and then precipitating out of solutionPXRD analysis of the solid recovered at 24 hours identified the precipitating entity as Form 2, suggesting that the 1:2 L-Lysine adduct convers to Form 2 by maturation in the aqueous buffer.

In contrast, solubility profiles of the adduct in sodium chloride 0.9% w/v (buffer v) and in deionised water (vi) are similar, increasing rapidly and then dropping after 1 hour of incubation. The peak solubility values measured at 1 hour are 29.81×10⁻³ mg·ml⁻¹ and 24.5×10⁻³ mg·ml⁻¹, respectively. It should be noted that the true peak may exceed these values, given that no intermediate measurements were made between 0 and 1 hours.

### Conclusions

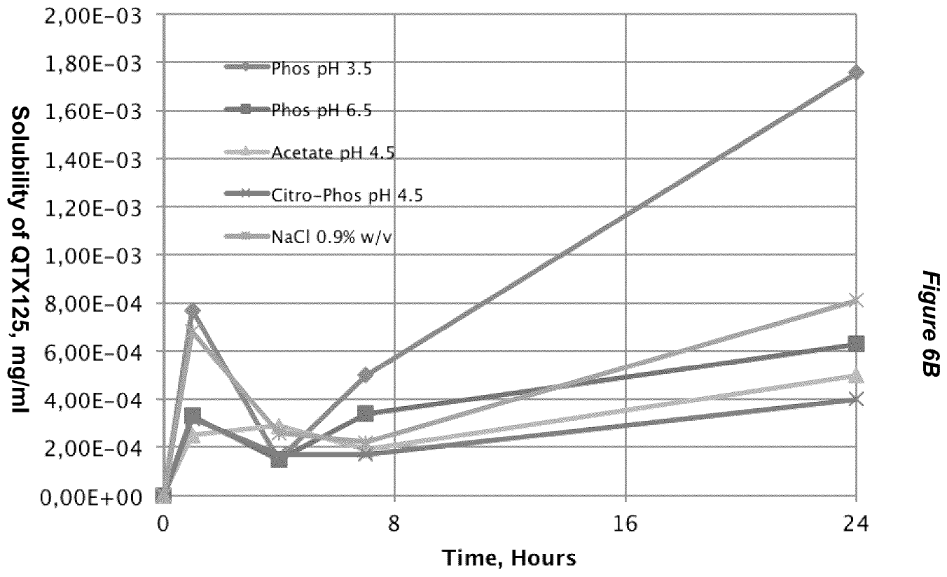

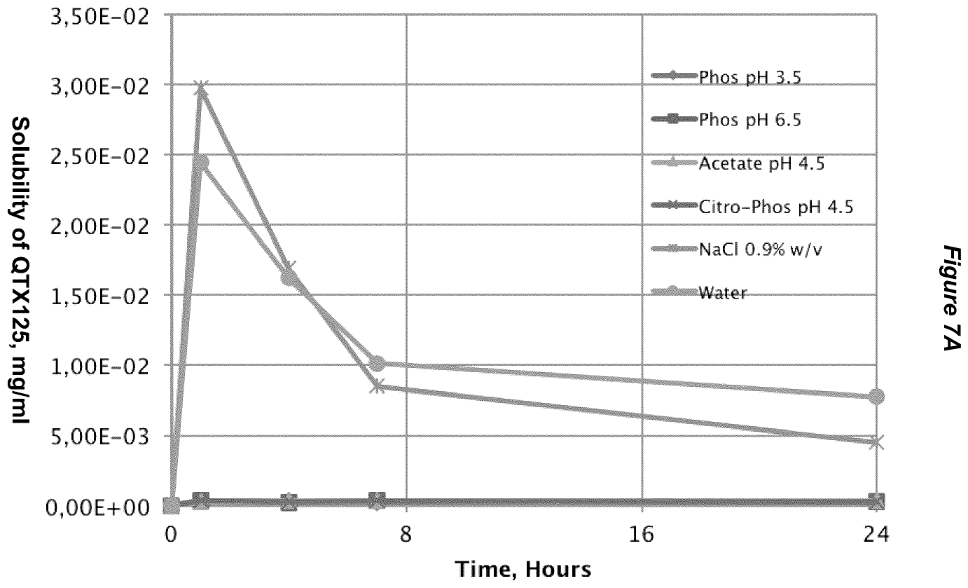

Form 2 displays its highest aqueous solubility in deionised water (buffer vi), reaching a peak of 0.02782 mg·ml⁻¹ following 7 hours of incubation. In contrast, the 1:2 L-Lysine adduct was unexpectedly observed to be soluble in both deionised water (buffer vi) and in sodium chloride solution (buffer v). Peak solubility was reached in following 1 hour of incubation, at 0.02450 mg·ml⁻¹ and 0.02981 mg·ml⁻¹, respectively. FIG. 8 provides a comparison of the solubility of the two QTX125 entities in deionised water (buffer vi). Both QTX125 entities appear to be poorly soluble in phosphate buffers pH 3.5 (i) and pH 6.5 (ii), in acetate buffer pH 4.5 (iii), and in citro-phosphate buffer pH 4.5 (iv), under the experimental conditions tested.

1. A crystalline form of a compound of formula I or an adduct thereof: 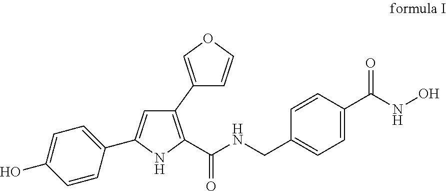 [SMILES: O=C(NO)c1ccc(CNC(=O)c2[nH]c(-c3ccc(O)cc3)cc2-c2ccoc2)cc1]
which is characterized by a powder x-ray diffraction pattern having peaks at 2θ=20.4°, 21.8°, 22.0°, 22.7°, and 23.9° (±0.3° 2θ).

2. The crystalline form of the compound of formula I or adduct thereof according to claim 1, further characterized by having a powder x-ray diffraction pattern having one or more peaks at 2θ selected from the group consisting of 9.0°, 11.2°, 11.7°, 12.6°, 15.1°, 18.0°, 24.3°, 26.1°, 26.6°, 30.5°, and 32.2° (±0.3° 2θ).

3. The crystalline form of the compound of formula I or adduct thereof according to claim 1, wherein the crystalline form has peaks at 2θ at:
9.0°, 12.6°, 13.0°, 14.2°, 16.5°, 16.9°, 20.4°, 21.8°, 22.0°, 22.7°, 23.9°, 26.6°, 30.50, and 32.20; or
5.6°, 8.2°, 11.2°, 11.7°, 13.0°, 15.1°, 16.9°, 17.7°, 18.0°, 20.4°, 21.8°, 22.0°, 22.7°, 23.9°, 24.30, and 26.1°.

4. The crystalline form of the compound of formula I or adduct thereof according to claim 1, having a powder x-ray diffraction pattern substantially similar to, or the same as, the powder x-ray diffraction pattern shown in FIG. 2A or FIG. 3A or FIG. 5A or FIG. 9.

5. The crystalline form of the compound of formula I according to claim 1, wherein the compound is Form 2.

6. The crystalline form of the compound of formula I or an adduct thereof according to claim 1, wherein the compound of formula I is adducted with at least one molecule of lysine.

7. The crystalline form of the of formula I or an adduct thereof according to claim 6, wherein the compound of formula I is adducted with two molecules of lysine.

8. The crystalline form of the of formula I or an adduct thereof according to claim 6, wherein the lysine is L-Lysine.

9. The crystalline form of the of formula I or an adduct thereof according to claim 1, which has a purity of at least 97%.

10. A pharmaceutical composition comprising a crystalline form of a compound of formula I or an adduct thereof according to claim 1 and a pharmaceutically acceptable excipient or carrier.

11. An in vitro complex, comprising a crystalline form of a compound of formula I or adduct thereof according to claim 1 and a histone deacetylase 6 (HDAC6).

12. A method of preparing a crystalline form of a compound of formula I according to claim 1, the method comprising the steps of:
(i) adding a compound of formula I to water to form a suspension;
(ii) heating the suspension;
(iii) adding one or more organic solvents before cooling; and
(iv) isolating a crystalline form of a compound of formula I or an adduct thereof.

13. A method of preparing a crystalline form of an adduct of a compound of formula I according to claim 1, the method comprising the steps of:
(i) adding a compound to be adducted to ethanol to form a first mixture;
(ii) adding a compound of formula I to water and one or more organic solvents to form a second mixture;
(iii) combining the first and second mixtures to form a composition;
(iv) cooling the composition; and
(v) isolating a crystalline form of an adduct of a compound of formula I.

14. (canceled)

15. A method of preparing a pharmaceutical composition comprising mixing a crystalline form of a compound of formula I or an adduct thereof according to claim 1 with a pharmaceutically acceptable excipient or carrier.

16. (canceled)

17. (canceled)

18. (canceled)

19. (canceled)

20. (canceled)

21. A method of treatment of a proliferative disease or an autoimmune disease, comprising administering a crystalline form of a compound of formula I or an adduct thereof according to claim 1, or a pharmaceutical composition thereof, to a mammalian subject in need thereof.

22. (canceled)

23. The method of treatment according to claim 21, wherein the proliferative disease is cancer.

24. (canceled)

25. (canceled)

26. (canceled)

27. A kit comprising a crystalline form of a compound of formula I or an adduct thereof according to claim 1 and pharmaceutically acceptable grade of water, buffer solution, or saline solution.
